(19) United States
(12) Patent Application Publication
(10) Pub. No.: US 2018/0214217 A1
(43) Pub. Date: Aug. 2, 2018

(54) SURGICAL INSTRUMENT WITH NAVIGATION WIRE INTERFACE FEATURES

(71) Applicants: Jephrey S. Rodriguez, Placentia, CA (US); Todd A. Veloni, Lake Forest, CA (US); Nawid E. Mehrzai, Mission Viejo, CA (US); Amit A. More, Irvine, CA (US)

(72) Inventors: Jephrey S. Rodriguez, Placentia, CA (US); Todd A. Veloni, Lake Forest, CA (US); Nawid E. Mehrzai, Mission Viejo, CA (US); Amit A. More, Irvine, CA (US)

(21) Appl. No.: 15/878,489

(22) Filed: Jan. 24, 2018

Related U.S. Application Data
(60) Provisional application No. 62/453,235, filed on Feb. 1, 2017.

(51) Int. Cl. A61B 34/20; A61M 25/09; A61M 1/00

(52) U.S. Cl. CPC A61B 34/20; A61M 25/0905; A61M 29/02; A61B 2034/2051; A61M 2205/502; A61M 1/0047

(57) ABSTRACT

An apparatus includes a cannula and a grip portion. The cannula includes a first lumen extending from a proximal end to a distal end of the cannula. The grip portion is secured to the proximal end of the cannula. The grip portion includes a second lumen, a transversely oriented vent port, a suction port, and a guidewire port. The second lumen is in communication with the first lumen. The transversely oriented vent port is configured to communicate with the second lumen to thereby vent the second lumen to atmosphere. The suction port is configured to communicate with the second lumen to thereby communicate suction to the distal end of the cannula via the first lumen. The guidewire port is configured to receive a guidewire. The guidewire port is further configured to communicate with the second lumen to thereby provide a path for the guidewire to reach the first lumen.

### PRIORITY

This application claims priority to U.S. Provisional Pat. App. No. 62/453,235, entitled “Surgical Instrument with Navigation Wire Interface Features,” filed Feb. 1, 2017, the disclosure of which is incorporated by reference herein.

### BACKGROUND

In some instances, it may be desirable to operate within or adjacent to an anatomical passageway of a patient, such as performing an incision of mucosa, removal of bone, or dilation of an anatomical passageway. Such operations may occur within anatomical passageways such as ostia of paranasal sinuses (e.g., to treat sinusitis), the larynx, the Eustachian tube, or other passageways within the ear, nose, or throat, etc. In addition to the above described operations, or similar operations, it may be desirable to apply suction and/or irrigation within or adjacent to an anatomical passageway before, during, or after the above described operations, or similar operations. One method of applying suction within or adjacent to an anatomical passageway of a patient involves obtaining a suction device having an elongate shaft defining a lumen terminating at an open distal end of the elongated shaft, where the lumen is in fluid communication with an external suction source. An operator may then insert the distal end of the elongate shaft within the nostril or mouth of a patient toward a desired location within the patient. With the distal end of the elongate shaft inserted within the patient, an operator may manipulate the suction device and/or suction source in order to remove extraneous and/or undesired matter near or within an anatomical passageway of a patient. Applying suction and/or irrigation during an operation may be beneficial for multiple purposes as will be apparent to those skilled in the art.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.) so as to superimpose the current location of the instrument on the preoperatively obtained images. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) mounted thereon are used to perform the procedure while the sensors send data to the computer indicating the current position of each surgical instrument. The computer correlates the data it receives from the instrument-mounted sensors with the digital map that was created from the preoperative tomographic scan. The tomographic scan images are displayed on a video monitor along with an indicator (e.g., cross hairs or an illuminated dot, etc.) showing the real time position of each surgical instrument relative to the anatomical structures shown in the scan images. In this manner, the surgeon is able to know the precise position of each sensor-equipped instrument by viewing the video monitor even if the surgeon is unable to directly visualize the instrument itself at its current location within the body.

Examples of electromagnetic IGS systems that may be used in ENT and sinus surgery include the InstaTrak ENT™ systems available from GE Medical Systems, Salt Lake City, Utah. Other examples of electromagnetic image guidance systems that may be modified for use in accordance with the present disclosure include but are not limited to the CARTO® 3 System by Biosense-Webster, Inc., of Diamond Bar, Calif.; systems available from Surgical Navigation Technologies, Inc., of Louisville, Colo.; and systems available from Calypso Medical Technologies, Inc., of Seattle, Wash.

When applied to functional endoscopic sinus surgery (FESS), balloon sinuplasty, and/or other ENT procedures, the use of image guidance systems allows the surgeon to achieve more precise movement and positioning of the surgical instruments than can be achieved by viewing through an endoscope alone. This is so because a typical endoscopic image is a spatially limited, 2 dimensional, line-of-sight view. The use of image guidance systems provides a real time, 3-dimensional view of all of the anatomy surrounding the operative field, not just that which is actually visible in the spatially limited, 2 dimensional, direct line-of-sight endoscopic view. As a result, image guidance systems may be particularly useful during performance of FESS, balloon sinuplasty, and/or other ENT procedures where a section and/or irrigation source may be desirable, especially in cases where normal anatomical landmarks are not present or are difficult to visualize endoscopically.

It may be desirable to provide features that further facilitate the use of an IGS navigation system and associated components in ENT procedures and other medical procedures. While several systems and methods have been made and used with respect to IGS and ENT surgery, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a schematic view of an exemplary sinus surgery navigation system;

FIG. 2 depicts a perspective view of the head of a patient, with components of the navigation system of FIG. 1;

FIG. 3 depicts a perspective view of an exemplary suction instrument;

FIG. 4 depicts a cross-sectional side view of the suction instrument of FIG. 3, taken along line 4-4 of FIG. 3;

FIG. 5 depicts a perspective view of the suction instrument of FIG. 3, with an adapter to receive a navigation guidewire;

FIG. 6 depicts a perspective view of another exemplary suction instrument, with an integral navigation guidewire port;

FIG. 7 depicts a top plan view of a grip portion of the suction instrument of FIG. 6;

FIG. 8 depicts a bottom plan view of the grip portion of FIG. 7;

FIG. 9 depicts a side elevational view of the grip portion of FIG. 7;

FIG. 10 depicts a cross-sectional side view of the grip portion of FIG. 7, taken along line 10-10 of FIG. 7;

FIG. 11 depicts a perspective view of another exemplary suction instrument, with an integral navigation guidewire port and guidewire locking feature;

FIG. 12 depicts a top plan view of a proximal portion of the suction instrument of FIG. 11;

FIG. 13 depicts a perspective view of the proximal portion of FIG. 12;

FIG. 14 depicts a cross-sectional top view of the proximal portion of FIG. 12, taken along line 14-14 of FIG. 15;

FIG. 15 depicts a cross-sectional side view of the proximal portion of FIG. 12, taken along line 15-15 of FIG. 12;

FIG. 16A depicts a cross-sectional top view of the proximal portion of FIG. 12, taken along line 14-14 of FIG. 15, with a navigation guidewire disposed therein, and with the locking feature in a locked state;

FIG. 16B depicts a cross-sectional top view of the proximal portion of FIG. 12, taken along line 14-14 of FIG. 15, with a navigation guidewire disposed therein, and with the locking feature in an unlocked state;

FIG. 17 depicts a perspective view of another exemplary suction instrument, with an integral navigation guidewire port and guidewire locking feature;

FIG. 18 depicts a top plan view of a grip portion of the suction instrument of FIG. 17;

FIG. 19 depicts a side elevational view of the grip portion of FIG. 18;

FIG. 20A depicts a cross-sectional perspective view of the grip portion of FIG. 18, taken along line 20-20 of FIG. 19, with the locking feature in a locked state;

FIG. 20B depicts a cross-sectional perspective view of the grip portion of FIG. 18, taken along line 20-20 of FIG. 19, with the locking feature in an unlocked state;

FIG. 21 depicts a perspective view of another exemplary suction instrument, with an integral navigation guidewire port and guidewire locking feature;

FIG. 22 depicts a top plan view of a grip portion of the suction instrument of FIG. 21;

FIG. 23 depicts a side elevational view of the grip portion of FIG. 22;

FIG. 24 depicts a cross-sectional side view of the grip portion of FIG. 22, taken along line 24-24 of FIG. 22;

FIG. 25A depicts a cross-sectional side view of the grip portion of FIG. 22, taken along line 24-24 of FIG. 22, with a navigation guidewire disposed therein, and with the locking feature in a locked state;

FIG. 25B depicts a cross-sectional side view of the grip portion of FIG. 22, taken along line 24-24 of FIG. 22, with a navigation guidewire disposed therein, and with the locking feature in an unlocked state;

FIG. 26 depicts a perspective view of another exemplary suction instrument, with an integral navigation guidewire port and guidewire locking feature;

FIG. 27 depicts a top plan view of a grip portion of the suction instrument of FIG. 26;

FIG. 28 depicts a perspective view of the grip portion of FIG. 27;

FIG. 29 depicts a cross-sectional top view of the grip portion of FIG. 27, taken along line 29-29 of FIG. 30;

FIG. 30 depicts a cross-sectional side view of the grip portion of FIG. 27, taken along line 30-30 of FIG. 27;

FIG. 31A depicts a cross-sectional top view of the grip portion of FIG. 27, taken along line 29-29 of FIG. 30, with a navigation guidewire disposed therein, and with the locking feature in a locked position; and

FIG. 31B depicts a cross-sectional top view of the grip portion of FIG. 27, taken along line 29-29 of FIG. 30, with a navigation guidewire disposed therein, and with the locking feature in an unlocked position.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Image Guided Surgery Navigation System

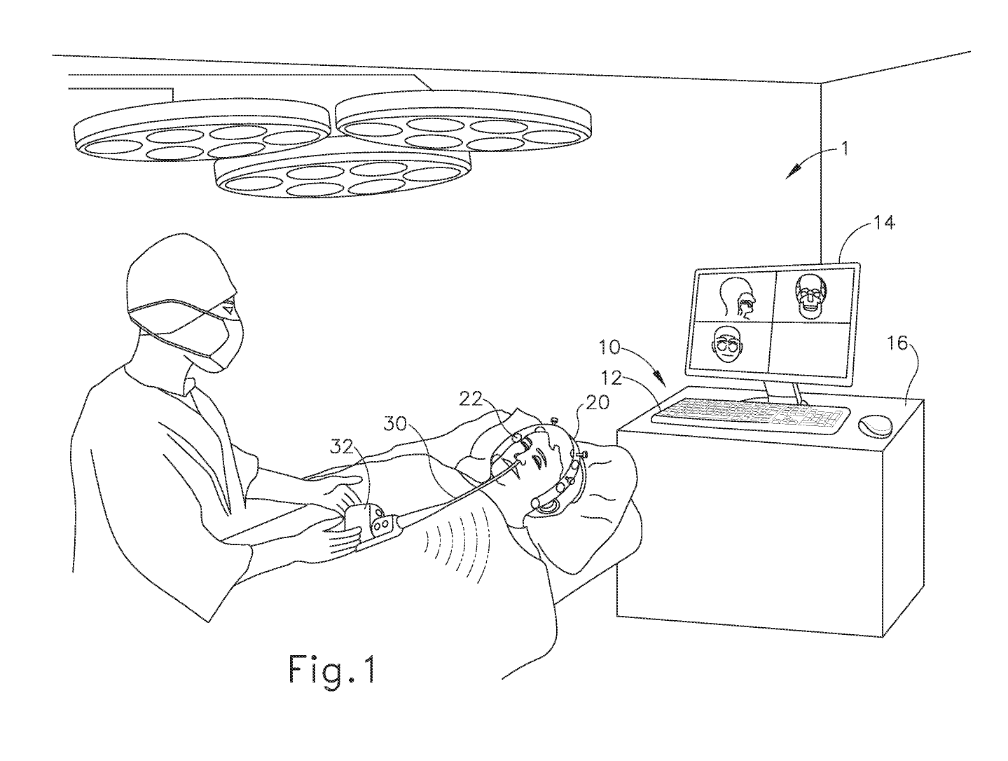

FIG. 1 shows an exemplary IGS navigation system (1) whereby an ENT procedure may be performed using IGS. In some instances, as will be described in greater detail below, IGS navigation system (1) is used during a procedure where suction adjacent to and/or within the procedure site is desired. However, it should be understood that IGS navigation system (1) may be readily used in various other kinds of procedures.

In addition to or in lieu of having the components and operability described herein IGS navigation system (1) may be constructed and operable in accordance with at least some of the teachings of U.S. Pat. No. 8,702,626, entitled “Guidewires for Performing Image Guided Procedures,” issued Apr. 22, 2014, the disclosure of which is incorporated by reference herein; U.S. Pat. No. 8,320,711, entitled “Anatomical Modeling from a 3-D Image and a Surface Mapping,” issued Nov. 27, 2012, the disclosure of which is incorporated by reference herein; U.S. Pat. No. 8,190,389, entitled “Adapter for Attaching Electromagnetic Image Guidance Components to a Medical Device,” issued May 29, 2012, the disclosure of which is incorporated by reference herein; U.S. Pat. No. 8,123,722, entitled “Devices, Systems and Methods for Treating Disorders of the Ear, Nose and Throat,” issued Feb. 28, 2012, the disclosure of which is incorporated by reference herein; and U.S. Pat. No. 7,720,521, entitled “Methods and Devices for Performing Procedures within the Ear, Nose, Throat and Paranasal Sinuses,” issued May 18, 2010, the disclosure of which is incorporated by reference herein.

Similarly, in addition to or in lieu of having the components and operability described herein, IGS navigation system (1) may be constructed and operable in accordance with at least some of the teachings of U.S. Pat. Pub. No. 2014/0364725, entitled “Systems and Methods for Performing Image Guided Procedures within the Ear, Nose, Throat and Paranasal Sinuses,” published Dec. 11, 2014, the disclosure of which is incorporated by reference herein; U.S. Pat. Pub. No. 2014/0200444, entitled “Guidewires for Performing Image Guided Procedures,” published Jul. 17, 2014, the disclosure of which is incorporated by reference herein; U.S. Pat. No. 9,198,736, entitled “Adapter for Attaching Electromagnetic Image Guidance Components to a Medical Device,” issued Dec. 1, 2015, the disclosure of which is incorporated by reference herein; U.S. Pat. Pub. No. 2011/0060214, entitled “Systems and Methods for Performing Image Guided Procedures within the Ear, Nose, Throat and Paranasal Sinuses,” published Mar. 10, 2011, the disclosure of which is incorporated by reference herein; U.S. Pat. No. 9,167,961, entitled “Methods and Apparatus for Treating Disorders of the Ear Nose and Throat,” issued Oct. 27, 2015, the disclosure of which is incorporated by reference herein; and U.S. Pat. Pub. No. 2007/0208252, entitled “Systems and Methods for Performing Image Guided Procedures within the Ear, Nose, Throat and Paranasal Sinuses,” published Sep. 6, 2007, the disclosure of which is incorporated by reference herein.

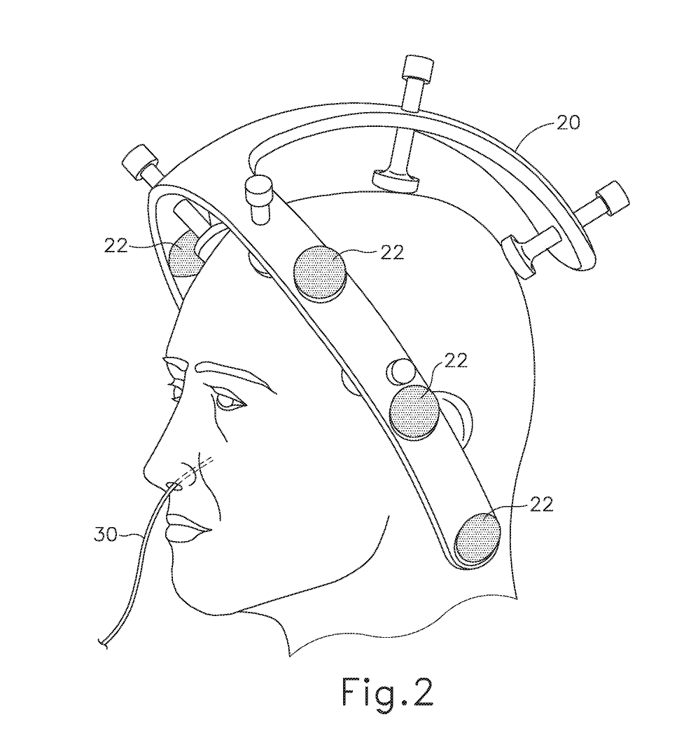

IGS navigation system (1) of the present example comprises a set of magnetic field generators (22). Before a surgical procedure begins, field generators (22) are fixed to the head of the patient. As best seen in FIG. 2, field generators (22) are incorporated into a frame (20), which is clamped to the head of the patient. While field generators (22) are secured to the head of the patient in this example, it should be understood that field generators (22) may instead be positioned at various other suitable locations and on various other suitable structures. By way of example only, field generators (22) may be mounted on an independent structure that is fixed to a table or chair on which the patient is positioned, on a floor-mounted stand that has been locked in position relative to the head of the patient, and/or at any other suitable location(s) and/or on any other suitable structure(s).

Field generators (22) are operable to generate an electromagnetic field around the head of the patient. In particular, field generators (22) are operated so as to transmit alternating magnetic fields of different frequencies into a region in proximity to frame (20). Field generators (22) thereby enable tracking of the position of a navigation guidewire (30) that is inserted into a nasal sinus of the patient and in other locations within the patient's head. Various suitable components that may be used to form and drive field generators (22) will be apparent to those of ordinary skill in the art in view of the teachings herein.

IGS navigation system (1) of the present example further comprises a processor (10), which controls field generators (22) and other elements of IGS navigation system (1). Processor (10) comprises a processing unit communicating with one or more memories. Processor (10) of the present example is mounted in a console (16), which comprises operating controls (12) that include a keypad and/or a pointing device such as a mouse or trackball. A physician uses operating controls (12) to interact with processor (10) while performing the surgical procedure.

Console (16) also connects to other elements of system (1). For instance, as shown in FIG. 1 a coupling unit (32) is secured to the proximal end of navigation guidewire (30). Coupling unit (32) of this example is configured to provide wireless communication of data and other signals between console (16) and navigation guidewire (30). In some versions, coupling unit (32) simply communicates data or other signals from navigation guidewire (30) to console (16) uni-directionally, without also communicating data or other signals from console (16). In some other versions, coupling unit (32) provides bidirectional communication of data or other signals between navigation guidewire (30) to console (16). While coupling unit (32) of the present example couples with console (16) wirelessly, some other versions may provide wired coupling between coupling unit (32) and console (16). Various other suitable features and functionality that may be incorporated into coupling unit (32) will be apparent to those of ordinary skill in the art in view of the teachings herein.

Processor (10) uses software stored in a memory of processor (10) to calibrate and operate system (1). Such operation includes driving field generators (22), processing data from navigational guidewire (30), processing data from operating controls (12), and driving display screen (14). The software may be downloaded to processor (10) in electronic form, over a network, for example, or it may, alternatively or additionally, be provided and/or stored on non-transitory tangible media, such as magnetic, optical, or electronic memory.

Processor (10) is further operable to provide video in real time via display screen (14), showing the position of the distal end of navigational guidewire (30) in relation to a video camera image of the patient's head, a CT scan image of the patient's head, and/or a computer generated three-dimensional model of the anatomy within and adjacent to the patient's nasal cavity. Display screen (14) may display such images simultaneously and/or superimposed on each other. Moreover, display screen (14) may display such images during the surgical procedure. Such displayed images may also include graphical representations of instruments that are inserted in the patient's head, such as navigational guidewire (30), such that the operator may view the virtual rendering of the instrument at its actual location in real time. Such graphical representations may actually look like the instrument or may be a much simpler representation such as a dot, crosshairs, etc. By way of example only, display screen (14) may provide images in accordance with at least some of the teachings of U.S. Pub. No. 2016/0008083, entitled “Guidewire Navigation for Sinuplasty,” published Jan. 14, 2016, the disclosure of which is incorporated by reference herein. In the event that the operator is also using an endoscope, the endoscopic image may also be provided on display screen (14). The images provided through display screen (14) may help guide the operator in maneuvering and otherwise manipulating instruments within the patient's head.

In the present example, navigational guidewire (30) includes one or more coils at the distal end of navigational guidewire (30). When such a coil is positioned within an electromagnetic field generated by field generators (22), movement of the coil within that magnetic field may generate electrical current in the coil, and this electrical current may be communicated along the electrical conduit(s) in navigational guidewire (30) and further to processor (10) via coupling unit (32). This phenomenon may enable IGS navigation system (1) to determine the location of the distal end of navigational guidewire (30) within a three-dimensional space as will be described in greater detail below. In particular, processor (10) executes an algorithm to calculate location coordinates of the distal end of navigational guidewire (30) from the position related signals of the coil(s) in navigational guidewire (30). While a coil is incorporated into navigational guidewire (30) to provide a navigational sensor in the present example, it should be understood that any other suitable components may be incorporated into navigational guidewire (30) to provide a navigational sensor.

In some instances, navigational guidewire (30) is used to generate a three-dimensional model of the anatomy within and adjacent to the patient's nasal cavity; in addition to being used to provide navigation for dilation catheter system (1) within the patient's nasal cavity. Alternatively, any other suitable device may be used to generate a three-dimensional model of the anatomy within and adjacent to the patient's nasal cavity before navigational guidewire (30) is used to provide navigation for dilation catheter system (1) within the patient's nasal cavity. By way of example only, a model of this anatomy may be generated in accordance with at least some of the teachings of U.S. Pub. No. 2016/0310042, entitled “System and Method to Map Structures of Nasal Cavity,” published Oct. 27, 2016, the disclosure of which is incorporated by reference herein. Still other suitable ways in which a three-dimensional model of the anatomy within and adjacent to the patient's nasal cavity may be generated will be apparent to those of ordinary skill in the art in view of the teachings herein. It should also be understood that, regardless of how or where the three-dimensional model of the anatomy within and adjacent to the patient's nasal cavity is generated, the model may be stored on console (16). Console (16) may thus render images of at least a portion of the model via display screen (14) and further render real-time video images of the position of navigational guidewire (30) in relation to the model via display screen (14).

II. Exemplary Suction Instrument Assembly

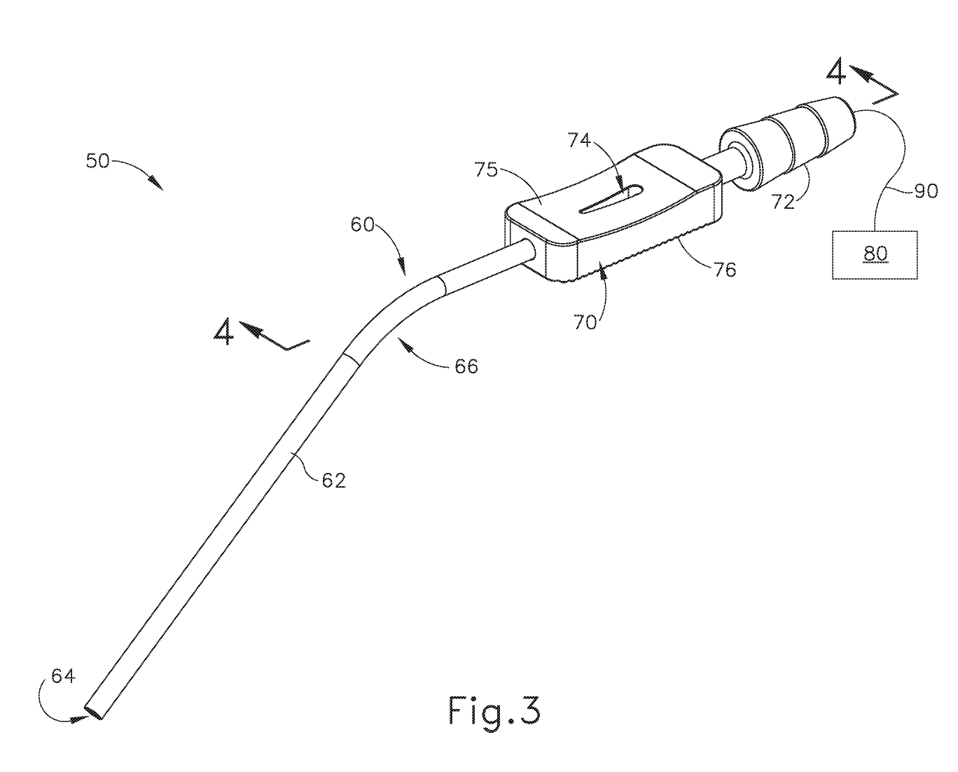

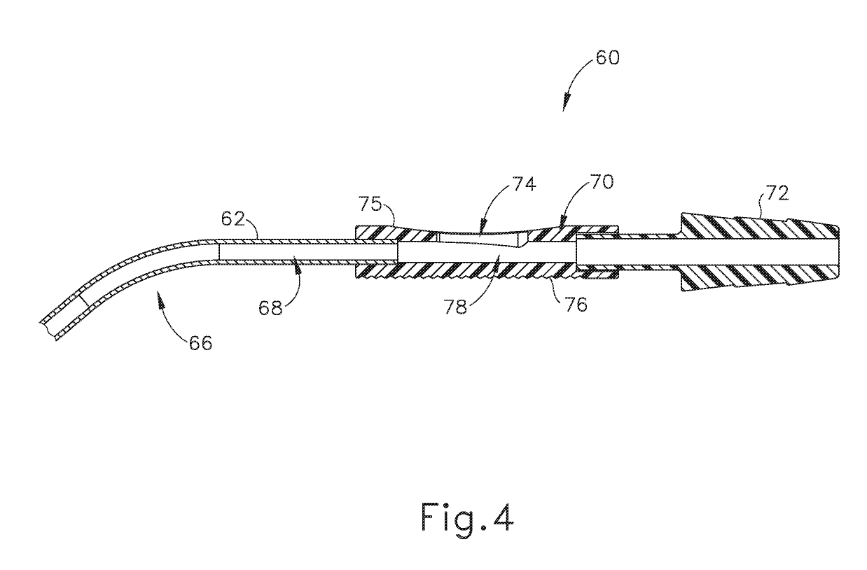

Various surgical procedures may warrant the use of a suction instrument in order to clear fluids and/or debris from the surgical field and/or from other sites within a patient. For instance, suction may be desirable in FESS procedures, sinuplasty procedures, and/or in various other ENT procedures. FIGS. 3-4 show an exemplary suction instrument assembly (50) that may be used to provide suction in such procedures. As shown, instrument assembly (50) includes a suction instrument (60) that is fluidly coupled with a suction source (80) via a conduit (90). Suction source (80) may comprise a vacuum pump and a fluid reservoir, among other components, as is known in the art. Suction source (80) is configured to provide enough suction to pull excess fluid and/or debris through suction instrument (60).

Suction instrument (60) of this example comprises an elongate cannula (62) extending distally from a grip portion (70). Cannula (62) has an open distal end (64) and a bent region (66) formed just distal to grip portion (70). Bent region (66) defines a bend angle that is selected to facilitate insertion of distal end (64) in a patient by an operator grasping grip portion (70). Various suitable bend angles that may be used will be apparent to those of ordinary skill in the art in view of the teachings herein.

In the present example, cannula (62) is rigid such that cannula (62) maintains the bend of bent region (66) and does not buckle during insertion into a patient's nasal cavity. By way of example only, cannula (62) may be formed of stainless steel (e.g., a stainless steel hypotube, etc.) and/or any other suitable rigid material. Also in the present example, cannula (62) defines a lumen (68) with a diameter of approximately 2.44 mm. Alternatively, any other suitable diameter may be used. It should also be understood that lumen (68) may have an elliptical cross-sectional profile or some other non-circular cross-sectional profile, if desired. A non-circular cross-sectional profile may provide additional clearance for other instruments to be positioned simultaneously in the same anatomical passageway (e.g., nasal cavity) with cannula (62).

Grip portion (70) of the present example includes a proximal suction conduit port (72) that is configured to couple with conduit (90). In the present example, port (72) has a barbed configuration to promote a secure fit with an elastomeric conduit (90), though it should be understood that various other kinds of configurations may be used for port (72). Grip portion (70) of the present example further includes a transverse vent opening (74) formed through an upper surface (75); and a lower surface (76). As best seen in FIG. 4, vent opening (74) is in fluid communication with a lumen (78) formed through grip portion (70). Vent opening (74) has a teardrop shape in the present example, though it should be understood that vent opening (74) may have any other suitable shape. By way of example only, the teardrop shape (or some other elongate shape) may enable the operator to selectively vary the amount of suction based on the longitudinal position of the operator's thumb (or other finger) on vent opening (74). Lumen (78) is further in fluid communication with port (72) and a lumen (68) of cannula (62). It should be understood that lumens (68, 78) cooperate to provide an unobstructed fluid path from port (72) to open distal end (64) of cannula (62).

Surfaces (75, 76) are configured to promote gripping of grip portion (70) by an operator. In particular, upper surface (75) provides a concave contour while lower surface (76) provides a series of ridges. By way of example only, an operator may grasp grip portion (70) by placing a thumb on upper surface (75) and the side of the index finger of the same hand on lower surface (76). The rectangular shape of grip portion (70) may provide the operator with substantial purchase on grip portion (70), while the configurations of surfaces (75, 76) may further secure the operator's grip.

During use of suction instrument assembly (50), the operator may grasp grip portion (70) and position distal end (64) of cannula (62) at a target site in a patient. In some such instances, suction source (80) remains in a constantly activated state. In those instances, the operator may leave vent opening (74) uncovered as the operator positions instrument (60) relative to the patient. This may result in suction source (80) drawing suction through vent opening (74) without drawing suction through open distal end (64). When the operator wishes to apply the suction to the target site in the patient via open distal end (64), the operator may simply cover vent opening (74) with the operator's thumb (or otherwise cover vent opening (74)). The operator may thus selectively cover and uncover vent opening during a procedure in order to selectively apply suction.

While the above and below examples are provided in the context of suction instruments, it should be understood that the same instruments (and variations thereof) may be used to provide fluid irrigation at a target site in a patient; or to provide various other kinds of functionality. The teachings herein are thus not limited to suction instruments and operations per se. Other suitable instruments and procedures in which the teachings herein may be applied will be apparent to those of ordinary skill in the art.

III. Exemplary Navigation Adapter for Suction Instrument

In some instances, it may be desirable to provide image guided navigation capabilities to a suction instrument like instrument (60). This may enable use of instrument (60) with IGS navigation system (1). Utilizing a IGS navigation system (1) in conjunction with a suction instrument like instrument (60) may allow the operator to provide better placement and tracking of distal end (64) of cannula (62) within the patient. In addition to providing navigation capabilities to a suction instrument like suction instrument (60), guidewire (30) of IGS navigation system (1) may be utilized by an operator in conjunction with instrument (60) in order to probe around mucosa of a patient without causing severe damage.

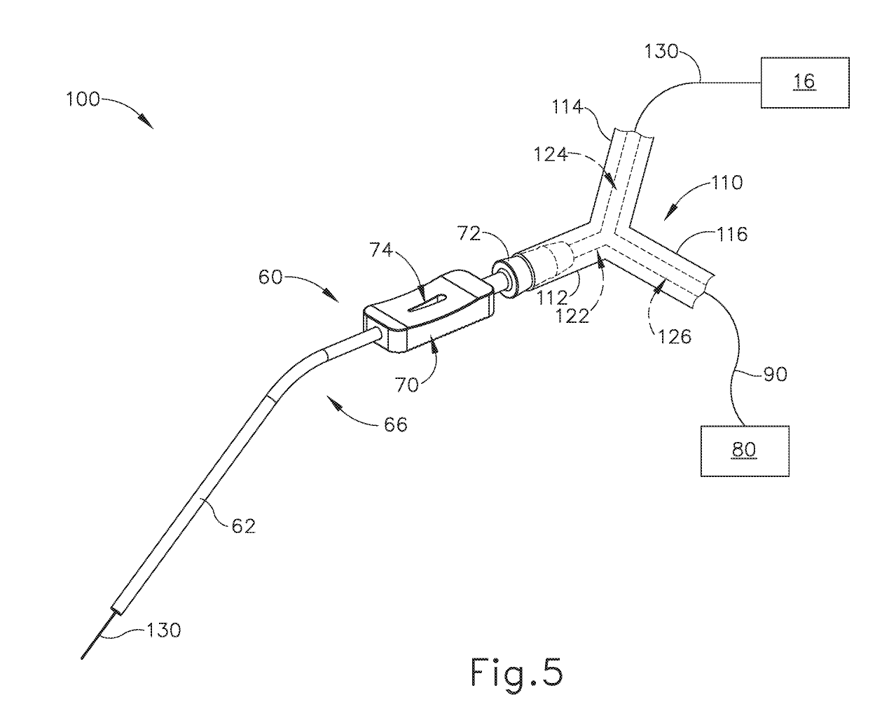

FIG. 5 shows an exemplary modified suction instrument assembly where instrument (60) has been modified to include an adapter (110). Adapter (110) of this example has a “Y” shape formed by an outlet (112), a first inlet (114), and a second inlet (116). Outlet (112) is coupled with conduit port (72) of grip portion (70). It should be understood that outlet (112) may be removably secured to conduit port (72), such that an operator may readily couple adapter (110) with grip portion (70) and decouple adapter (110) from grip portion (70) as desired. Outlet (112) defines an outlet lumen (122) that is in fluid communication with lumen (78) of grip portion (70).

First inlet (114) defines a first inlet lumen (124), which is in communication with outlet lumen (122). A navigation guidewire (130) is slidably disposed in first inlet lumen (124). Navigation guidewire (130) continues through outlet lumen (122), through lumen (78) of grip portion (70), and further through lumen (68) of cannula (62), such that the distal end of navigation guidewire (130) protrudes distally from distal end (64) of cannula (62). The proximal end of guidewire (130) is coupled with console (16). It should be understood that guidewire (130) of this example is configured and operable just like guidewire (30) described above. Thus, an operator may use IGS navigation system (1) to determine the location of the distal end of guidewire (130) within a patient, to thereby determine the location of distal end (64) of cannula (62) in the patient. Moreover, the operator may freely slide guidewire (130) within lumens (68, 78, 122, 124), to thereby facilitate probing with the distal end of guidewire (130).

Second inlet (116) defines a second inlet lumen (126), which is also in communication with outlet lumen (122). Conduit (90) is coupled with second inlet (116). Conduit (90) is also coupled with suction source (80). Conduit (90) thus provides a path for suction from suction source (80) to second inlet lumen (126). When the operator covers vent opening (74) as described above, the suction from suction source (80) is further communicated to distal end (64) of cannula (62) via lumens (68, 78, 122, 126). It should therefore be understood that lumens (68, 78, 122) provide a path for communication of suction and guidewire (130). It should also be understood that the diameters of lumens (68, 78, 122) are large enough to enable an effective amount of suction to reach distal end (64) even while guidewire (130) is disposed in lumens (68, 78, 122).

In some versions, first inlet (114) further includes a seal. Such a seal may substantially prevent atmospheric air from being drawn in through first inlet lumen (124) while suction source (80) is being activated; while still permitting guidewire (130) to slide substantially freely through first inlet lumen (124). In some variations, the seal provides friction against guidewire (130), such that the seal substantially prevents inadvertent translation of guidewire (130) through inlet lumen (124) while still permitting intended translation of guidewire (130) through inlet lumen (124). Various suitable forms that such a seal may take will be apparent to those of ordinary skill in the art in view of the teachings herein.

IV. Exemplary Suction Instrument with Integral Navigation Guidewire Port

As noted above, it may be desirable in some instances to provide image guided navigation capabilities to a suction instrument like instrument (60). It may also be desirable to provide such capabilities without compromising the ergonomics of the suction instrument. To accomplish this, it may be desirable to provide a suction instrument with an integral navigation guidewire port.

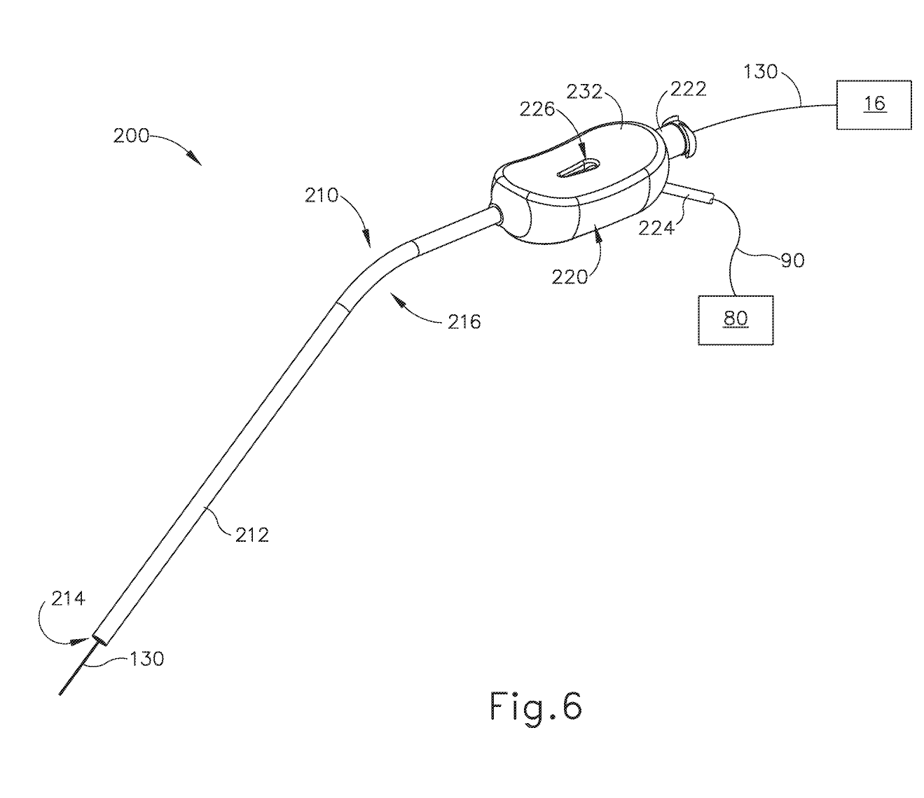

FIG. 6 shows an exemplary suction instrument assembly (200) that includes a suction instrument (210) having an integral navigation guidewire port (222). Suction instrument assembly (200) of this example further includes console (16), navigation guidewire (130), suction source (80), and conduit (90), each of which is described above. Suction instrument (210) of the present example includes an elongate cannula (212) extending distally from a grip portion (220). Cannula (212) has an open distal end (214) and a bent region (216) formed just distal to grip portion (220). Bent region (216) defines a bend angle that is selected to facilitate insertion of distal end (214) in a patient by an operator grasping grip portion (220). Various suitable bend angles that may be used will be apparent to those of ordinary skill in the art in view of the teachings herein.

In the present example, cannula (212) is rigid such that cannula (212) maintains the bend of bent region (216) and does not buckle during insertion into a patient's nasal cavity. By way of example only, cannula (212) may be formed of stainless steel (e.g., a stainless steel hypotube, etc.) and/or any other suitable rigid material. Also in the present example, cannula (212) defines a lumen with a diameter of approximately 2.44 mm. Alternatively, any other suitable diameter may be used. It should also be understood that the lumen of cannula (212) may have an elliptical cross-sectional profile or some other non-circular cross-sectional profile, if desired. A non-circular cross-sectional profile may provide additional clearance for other instruments to be positioned simultaneously in the same anatomical passageway (e.g., nasal cavity) with cannula (212).

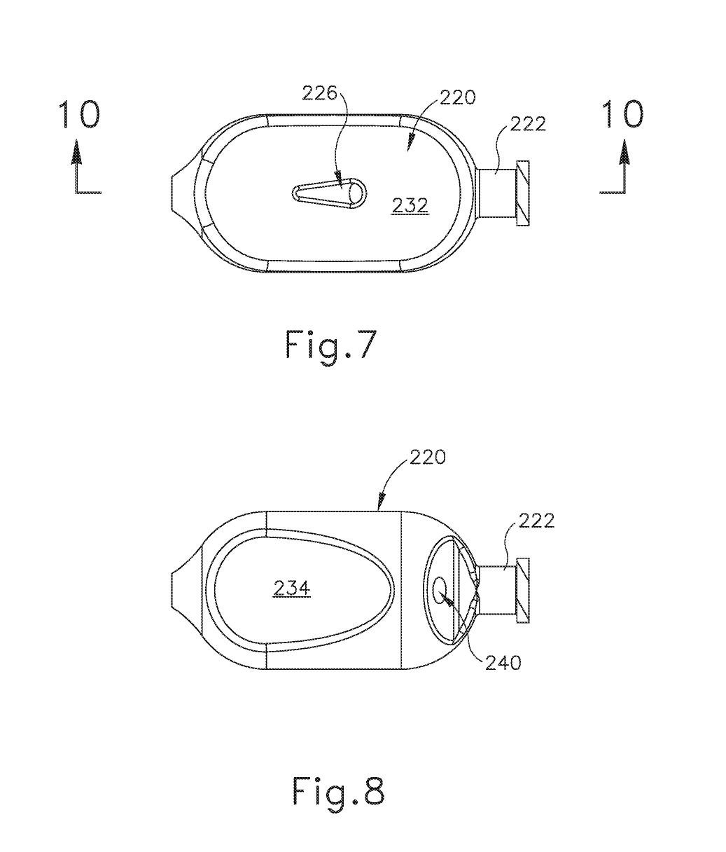

FIGS. 7-10 show grip portion (220) in greater detail. It should be understood that the rest of the components of suction instrument assembly (200) are omitted from FIGS. 7-10, for clarity. Grip portion (220) of the present example includes a proximal suction conduit port (224) that is configured to couple with conduit (90). Port (224) is inserted in an opening (240) that is formed in grip portion (220), as seen in FIGS. 8 and 10. In some versions, port (224) has a barbed configuration to promote a secure fit with an elastomeric conduit (90), though it should be understood that various other kinds of configurations may be used for port (224), Grip portion (220) of the present example further includes a transverse vent opening (226) formed through an upper surface (232); and a lower surface (234). As best seen in FIG. 10, vent opening (226) is in fluid communication with a lumen (228) formed through grip portion (220). As best seen in FIG. 7, vent opening (226) has a teardrop shape in the present example, though it should be understood that vent opening (226) may have any other suitable shape. By way of example only, the teardrop shape (or some other elongate shape) may enable the operator to selectively vary the amount of suction based on the longitudinal position of the operator's thumb (or other finger) on vent opening (226). Lumen (228) is further in fluid communication with suction port opening (240) and a lumen (not shown) of cannula (212). It should be understood that lumen (228) of grip portion (220) and the lumen of cannula (212) cooperate to provide an unobstructed fluid path from port (224) to open distal end (214) of cannula (212).

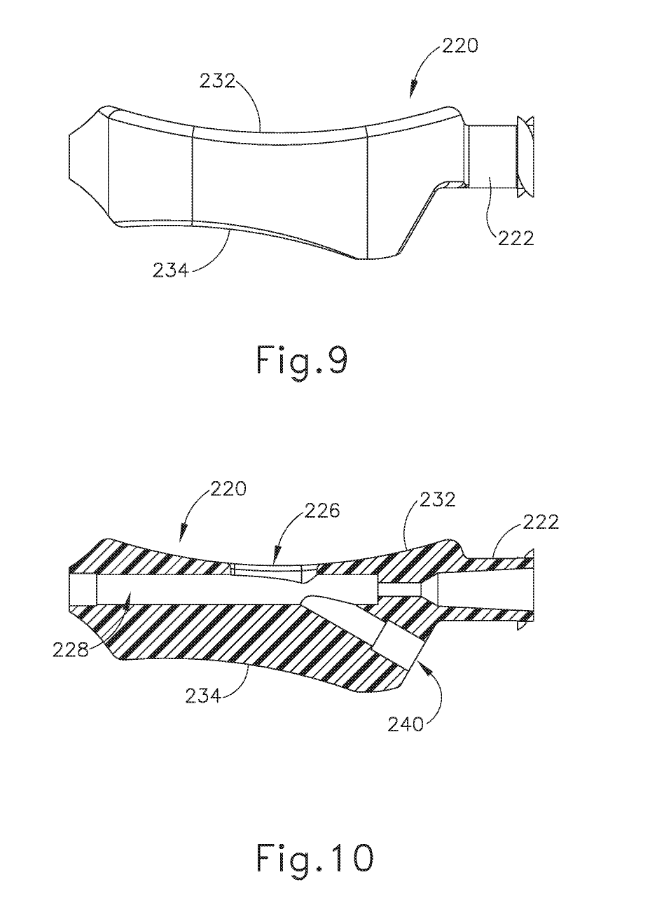

Surfaces (232, 234) are configured to promote gripping of grip portion (220) by an operator. In particular, as best seen in FIG. 9, each surface (232, 234) provides a respective concave contour. By way of example only, an operator may grasp grip portion (220) by placing a thumb on upper surface (232) and the side of the index finger of the same hand on lower surface (234). The oblong shape of grip portion (220) may provide the operator with substantial purchase on grip portion (220), while the configurations of surfaces (232, 234) may further secure the operator's grip.

Grip portion (220) of the present example also includes navigation guidewire port (222), as noted above. As best seen in FIG. 10, navigation guidewire port (222) is in communication with lumen (228) of grip portion (220). Navigation guidewire (130) is slidably disposed in navigation guidewire port (222). Navigation guidewire (130) continues through lumen (228) of grip portion (220), and further through the lumen of cannula (212), such that the distal end of navigation guidewire (130) protrudes distally from distal end (214) of cannula (212). The proximal end of guidewire (130) is coupled with console (16). In some versions, guidewire port (222) is fitted with a feature that is operable to secure the position of guidewire (130) relative to grip portion (220). By way of example only, guidewire port (222) may be fitted with a Touhy Borst leur adapter that is operable to secure the position of guidewire (130) relative to grip portion (220

It should be understood that guidewire (130) of this example is configured and operable just like guidewire (30) described above. Thus, an operator may use IGS navigation system (1) to determine the location of the distal end of guidewire (130) within a patient, to thereby determine the location of distal end (214) of cannula (212) in the patient. Moreover, the operator may freely slide guidewire (130) within lumen (228) of grip portion (220) and the lumen of cannula (212), to thereby facilitate probing with the distal end of guidewire (130). The diameters of lumen (228) and the lumen of cannula (212) are large enough to enable an effective amount of suction to reach distal end (214) even while guidewire (130) is disposed in lumen (228) of grip portion (220) and the lumen of cannula (212).

In the present example, navigation guidewire port (222) is coaxially aligned with the longitudinal axis of lumen (228) of grip portion (220); while opening (240) and conduit port (224) are oriented obliquely relative to the longitudinal axis of lumen (228) of grip portion (220). In some other versions, these relationships are reversed. In other words, navigation guidewire port (222) may be obliquely oriented relative to the longitudinal axis of lumen (228) of grip portion (220); while opening (240) and conduit port (224) may be coaxially aligned with the longitudinal axis of lumen (228) of grip portion (220). Other suitable orientations and arrangements will be apparent to those of ordinary skill in the art in view of the teachings herein.

During use of suction instrument assembly (200), the operator may grasp grip portion (220) and position distal end (214) of cannula (212) at a target site in a patient. In some such instances, suction source (80) remains in a constantly activated state. In those instances, the operator may leave vent opening (226) uncovered as the operator positions instrument (210) relative to the patient. This may result in suction source (80) drawing suction through vent opening (226) without drawing suction through open distal end (214). During the act of positioning instrument (210), the operator may rely on navigation guidewire (130) and the rest of IGS navigation system (1) to provide image guidance as described above. When the operator reaches the target site and wishes to apply the suction to the target site in the patient via open distal end (214), the operator may simply cover vent opening (226) with the operator's thumb (or otherwise cover vent opening (226)). The operator may thus selectively cover and uncover vent opening during a procedure in order to selectively apply suction. Moreover, the operator may use navigation guidewire (130) and the rest of IGS navigation system (1) to provide image guidance to suction instrument (210) before, during, and/or after the suctioning procedure.

V. Exemplary Suction Instrument with Integral Navigation Guidewire Port and Guidewire Locking Feature

In versions of suction instruments that include an integral navigation guidewire port, it may be desirable to provide a feature that enables the operator to selectively lock the longitudinal position of the navigation guidewire relative to the suction instrument. This may ensure that the distal end of the navigation guidewire maintains a consistent position relative to the distal end of the cannula of the suction instrument. In addition to selectively locking the longitudinal position of a navigation guidewire relative to a suction instrument, a locking feature may further provide a seal that prevents the loss of suction through the navigation guidewire port. The following description provides several merely illustrative examples of suction instruments that include features that are operable to selectively lock the longitudinal position of the navigation guidewire relative to the suction instrument. Other variations will be apparent to those of ordinary skill in the art in view of the teachings herein.

A. First Exemplary Guidewire Locking Feature

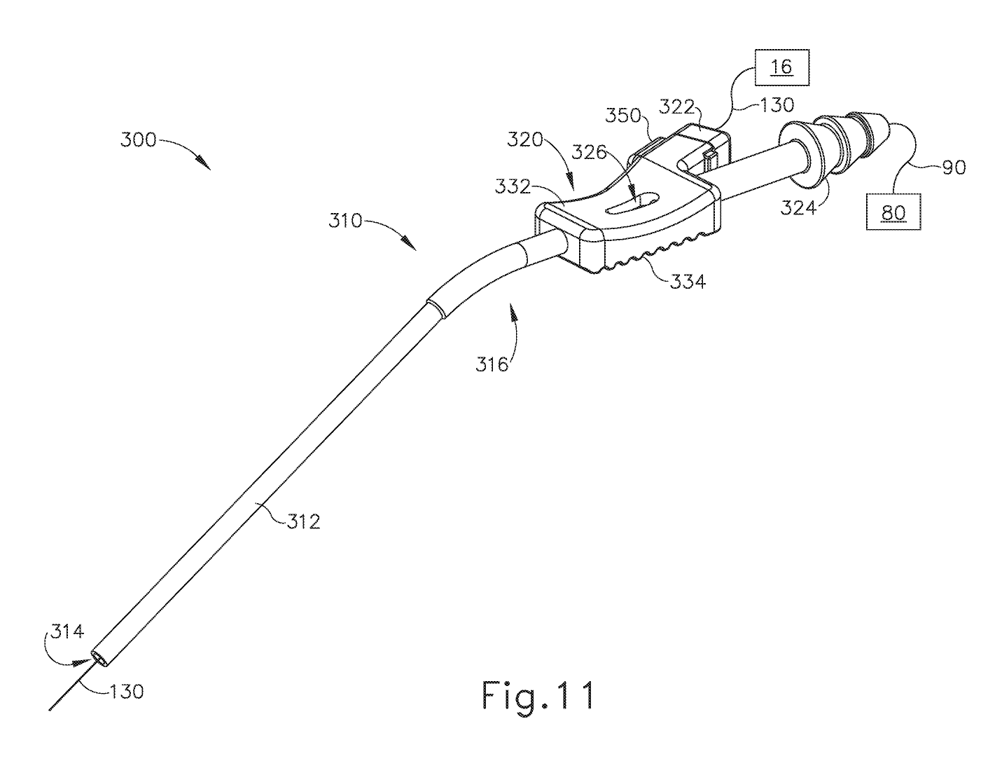

FIG. 11 shows an exemplary suction instrument assembly (300) that includes a suction instrument (310) having an integral navigation guidewire port (322). Suction instrument assembly (300) of this example further includes console (16), navigation guidewire (130), suction source (80), and conduit (90), each of which is described above. Suction instrument (310) of the present example includes an elongate cannula (312) extending distally from a grip portion (320). Cannula (312) has an open distal end (314) and a bent region (316) formed just distal to grip portion (320). Bent region (316) defines a bend angle that is selected to facilitate insertion of distal end (314) in a patient by an operator grasping grip portion (320). Various suitable bend angles that may be used will be apparent to those of ordinary skill in the art in view of the teachings herein.

In the present example, cannula (312) is rigid such that cannula (312) maintains the bend of bent region (316) and does not buckle during insertion into a patient's nasal cavity. By way of example only, cannula (312) may be formed of stainless steel (e.g., a stainless steel hypotube, etc.) and/or any other suitable rigid material. Also in the present example, cannula (312) defines a lumen with a diameter of approximately 2.44 mm. Alternatively, any other suitable diameter may be used. It should also be understood that the lumen of cannula (312) may have an elliptical cross-sectional profile or some other non-circular cross-sectional profile, if desired. A non-circular cross-sectional profile may provide additional clearance for other instruments to be positioned simultaneously in the same anatomical passageway (e.g., nasal cavity) with cannula (312).

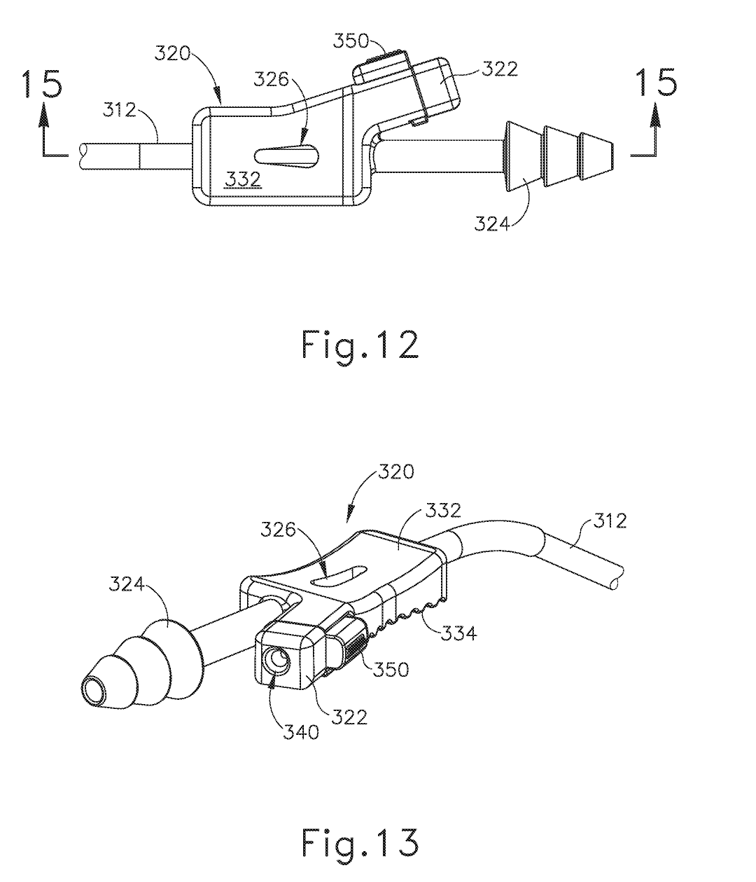

FIGS. 12-16B show grip portion (320) in greater detail. Grip portion (320) of the present example includes a proximal suction conduit port (324) that is configured to couple with conduit (90). In the present example, port (324) has a barbed configuration to promote a secure fit with an elastomeric conduit (90), though it should be understood that various other kinds of configurations may be used for port (324). Grip portion (320) of the present example further includes a transverse vent opening (326) formed through an upper surface (332); and a lower surface (334). As best seen in FIG. 15, vent opening (326) is in fluid communication with a lumen (328) formed through grip portion (320). As best seen in FIG. 12, vent opening (326) has a teardrop shape in the present example, though it should be understood that vent opening (326) may have any other suitable shape. By way of example only, the teardrop shape (or some other elongate shape) may enable the operator to selectively vary the amount of suction based on the longitudinal position of the operator's thumb (or other finger) on vent opening (326). Lumen (328) is further in fluid communication with suction conduit port (324) and a lumen (not shown) of cannula (312). It should be understood that lumen (328) of grip portion (320) and the lumen of cannula (312) cooperate to provide an unobstructed fluid path from port (324) to open distal end (314) of cannula (312).

Surfaces (332, 334) are configured to promote gripping of grip portion (320) by an operator. In particular, as best seen in FIGS. 13 and 15, upper surface (332) provides a concave contour; while lower surface (334) provides a set of ridges. By way of example only, an operator may grasp grip portion (320) by placing a thumb on upper surface (332) and the side of the index finger of the same hand on lower surface (334). The generally rectangular shape of grip portion (320) may provide the operator with substantial purchase on grip portion (320), while the configurations of surfaces (332, 334) may further secure the operator's grip.

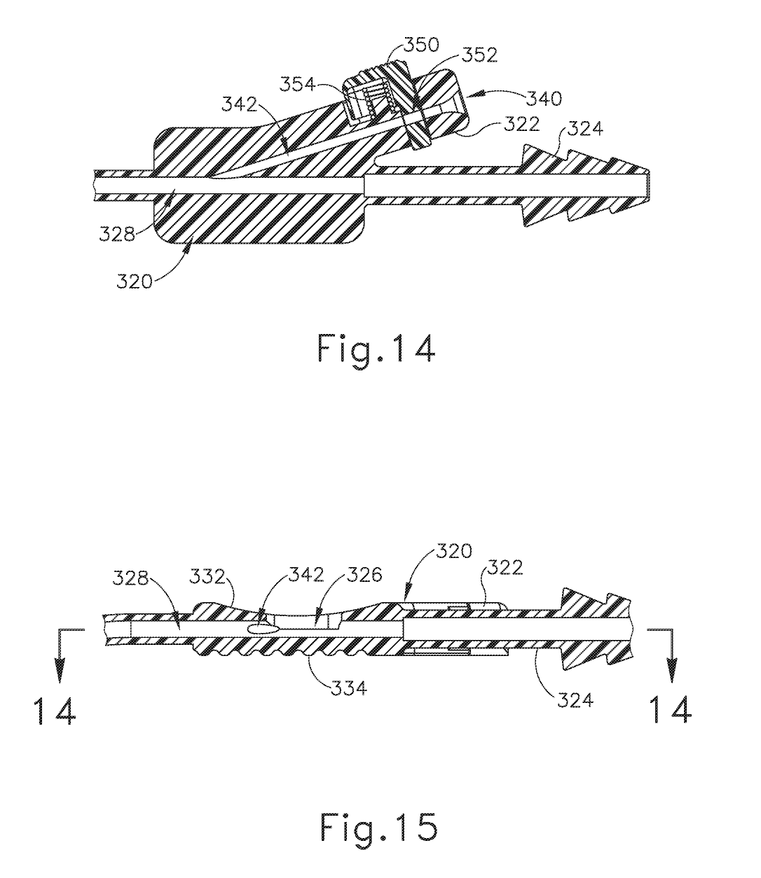

Grip portion (320) of the present example also includes navigation guidewire port (322), as noted above. As best seen in FIG. 14, navigation guidewire port (322) is in the form of an arm that defines an opening (340) leading to a lumen (342). The outer region of opening (340) is funnel-shaped or tapered to facilitate insertion of navigation guidewire (130) into opening (340) and lumen (342). Lumen (342) of navigation guidewire port (322) is in communication with lumen (328) of grip portion (320). Navigation guidewire (130) is slidably disposed in navigation guidewire port (322). Navigation guidewire (130) continues through lumen (328) of grip portion (320), and further through the lumen of cannula (312), such that the distal end of navigation guidewire (130) protrudes distally from distal end (314) of cannula (312). The proximal end of guidewire (130) is coupled with console (16). As also seen in FIG. 14, lumen (342) reaches lumen (328) at a point that is distal to the location of vent opening (326). This positioning may prevent guidewire (130) from inadvertently exiting vent opening (326) while guidewire (130) is being initially fed into grip portion (320).

Grip portion (320) of the present example further includes a locking member (350), which is in the form of a push-button in the present example. Locking member (350) is secured to guidewire port (322) and is configured to translate transversely relative to guidewire port (322) through a limited range of motion. Locking member (350) defines a lumen (352) that is configured to selectively align with lumen (342) of guidewire port (322). A coil spring (354) is configured to urge locking member (350) toward the position shown in FIG. 14, where lumen (352) is not aligned with lumen (342). While a coil spring (354) is used to provide a resilient bias in the present example, it should be understood that any other suitable kind of resilient member may be used.

In the present example, guidewire port (322) and locking member (350) are positioned such that locking member (350) is laterally offset from the vertical plane that passes through the entire length of cannula (312) and vent opening (326). Due to this positioning, an operator may tend to use a finger (other than the thumb) of the hand that grasps grip portion (320) to actuate locking member (350). The operator may this keep vent opening (326) covered with their thumb while actuating locking member (350). Of course, an operator may actuate locking member (350) in any other suitable fashion.

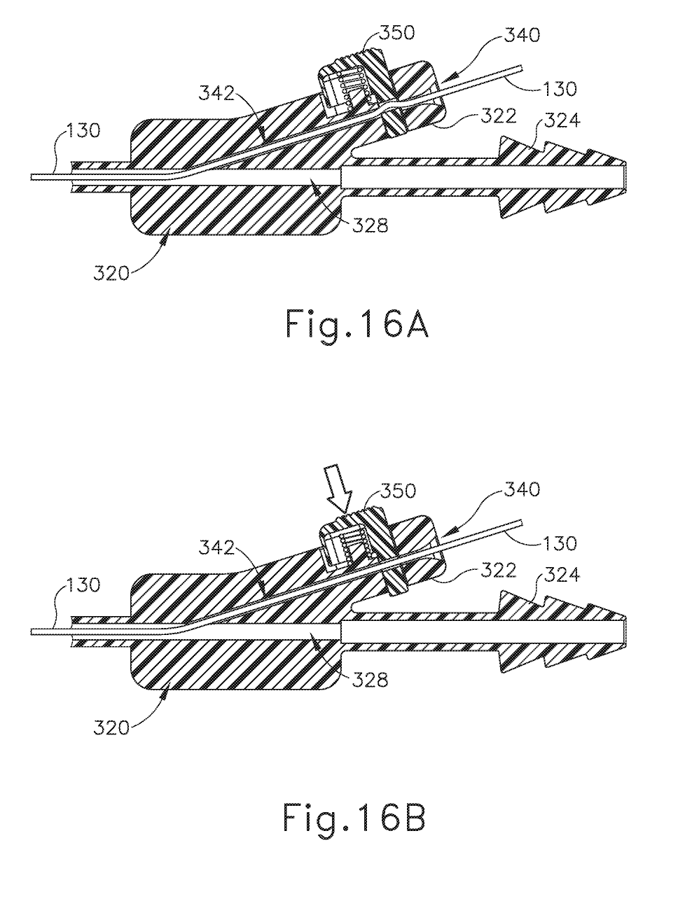

FIGS. 16A-16B show the relationship between locking member (350) and navigation guidewire (130). In FIG. 16A, locking member (350) is in an outward position to provide a locked state. In this position, lumen (352) of locking member (350) is not aligned with lumen (342) of guidewire port (322). Since navigation guidewire (130) is disposed in both lumens (342, 352), the non-alignment of lumens (342, 352) provides a pinching effect on navigation guidewire (130). This pinching effect creates friction on navigation guidewire (130), thereby substantially securing the longitudinal position of navigation guidewire (130) relative to suction instrument (310). In other words, navigation guidewire (130) is in a locked state in FIG. 16A.

If the operator wishes to translate navigation guidewire (130) relative to suction instrument (310) (i.e., to advance or retract the distal end of navigation guidewire (130) relative to distal end (314) of cannula (312)), the operator may press locking member (350) inwardly, against the bias of coil spring (354). This eventually results in the configuration shown in FIG. 16B. In this state, lumens (342, 352) are aligned with each other, thereby relieving the pinching effect on navigation guidewire (130). Navigation guidewire (130) is then capable of sliding freely relative to suction instrument (310), such that navigation guidewire (130) is in an unlocked state. Once the operator is satisfied with the adjusted longitudinal position of navigation guidewire (130) relative to suction instrument (310), the operator may release locking member (350). Coil spring (354) will then return locking member (350) back to the position shown in FIG. 16A, thereby locking the adjusted longitudinal position of navigation guidewire (130) relative to suction instrument (310). Locking member (350) and guidewire port (322) may include structural features that cooperate to restrict the movement of locking member (350) to the range of motion depicted between FIGS. 16A-16B.

It should be understood that guidewire (130) of this example is configured and operable just like guidewire (30) described above. Thus, an operator may use IGS navigation system (1) to determine the location of the distal end of guidewire (130) within a patient, to thereby determine the location of distal end (314) of cannula (312) in the patient. Moreover, after transitioning locking member (350) to the unlocked position, the operator may freely slide guidewire (130) within lumen (328) of grip portion (320) and the lumen of cannula (312), to thereby facilitate probing with the distal end of guidewire (130). The diameters of lumen (328) and the lumen of cannula (312) are large enough to enable an effective amount of suction to reach distal end (314) even while guidewire (130) is disposed in lumen (328) of grip portion (320) and the lumen of cannula (312).

In the present example, and as best seen in FIG. 14, navigation guidewire port (322) is obliquely oriented relative to the longitudinal axis of lumen (328) of grip portion (320); while conduit port (324) is coaxially aligned with the longitudinal axis of lumen (328) of grip portion (320). In some other versions, these relationships are reversed. In other words, navigation guidewire port (322) may be coaxially aligned with the longitudinal axis of lumen (328) of grip portion (320); while conduit port (324) is oriented obliquely relative to the longitudinal axis of lumen (328) of grip portion (320). Other suitable orientations and arrangements will be apparent to those of ordinary skill in the art in view of the teachings herein.

During use of suction instrument assembly (300), the operator may grasp grip portion (320) and position distal end (314) of cannula (312) at a target site in a patient. In some such instances, suction source (80) remains in a constantly activated state. In those instances, the operator may leave vent opening (326) uncovered as the operator positions instrument (310) relative to the patient. This may result in suction source (80) drawing suction through vent opening (326) without drawing suction through open distal end (314). During the act of positioning instrument (310), the operator may rely on navigation guidewire (130) and the rest of IGS navigation system (1) to provide image guidance as described above. This may include manipulation of locking member (350) to enable adjustment of the longitudinal position of navigation guidewire (130) relative to suction instrument (310).

When the operator reaches the target site and wishes to apply the suction to the target site in the patient via open distal end (314), the operator may simply cover vent opening (326) with the operator's thumb (or otherwise cover vent opening (326)). The operator may thus selectively cover and uncover vent opening during a procedure in order to selectively apply suction. Moreover, the operator may use navigation guidewire (130) and the rest of IGS navigation system (1) to provide image guidance to suction instrument (310) before, during, and/or after the suctioning procedure. This may include using locking member (350) to selectively lock and unlock the longitudinal position of navigation guidewire (130) relative to suction instrument (310).

In the present example, locking member (350) is configured to cooperate with guidewire port (322) and navigation guidewire (130) to substantially seal guidewire port (322) when locking member (350) is in the locked position shown in FIG. 16A. Thus, when navigation guidewire (130) and locking member (350) are in a locked state, suction instrument (310) will not lose meaningful suction through guidewire port (322). Moreover, locking member (350) will prevent loss of suction through guidewire port (322) when a navigation guidewire (130) is not disposed in guidewire port (322). Other features that may be used to prevent loss of suction will be apparent to those of ordinary skill in the art in view of the teachings herein.

B. Second Exemplary Guidewire Locking Feature

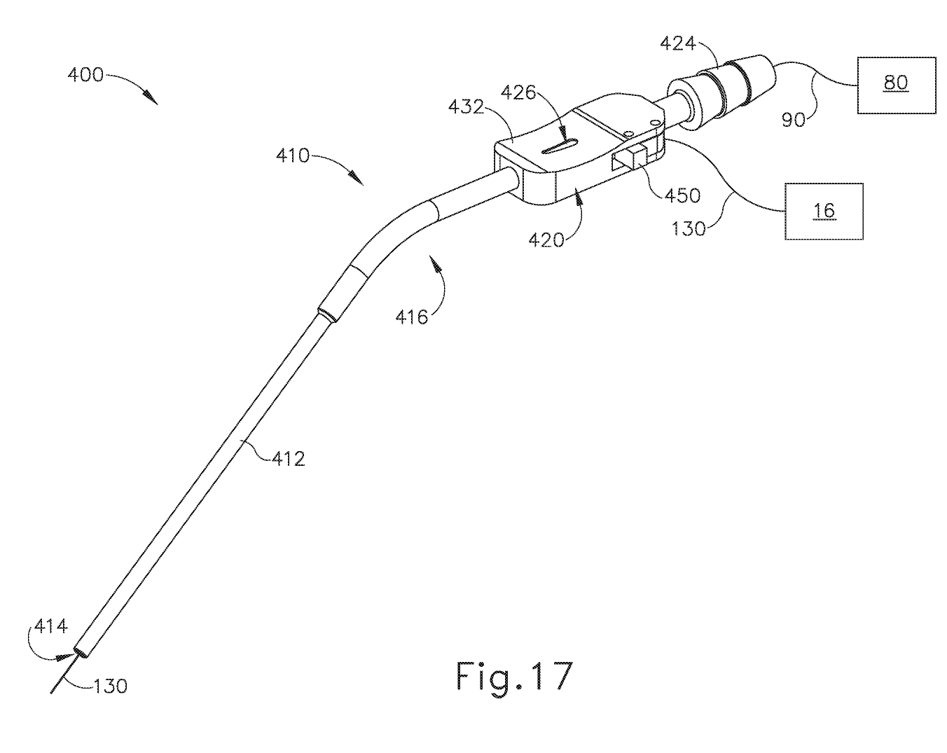

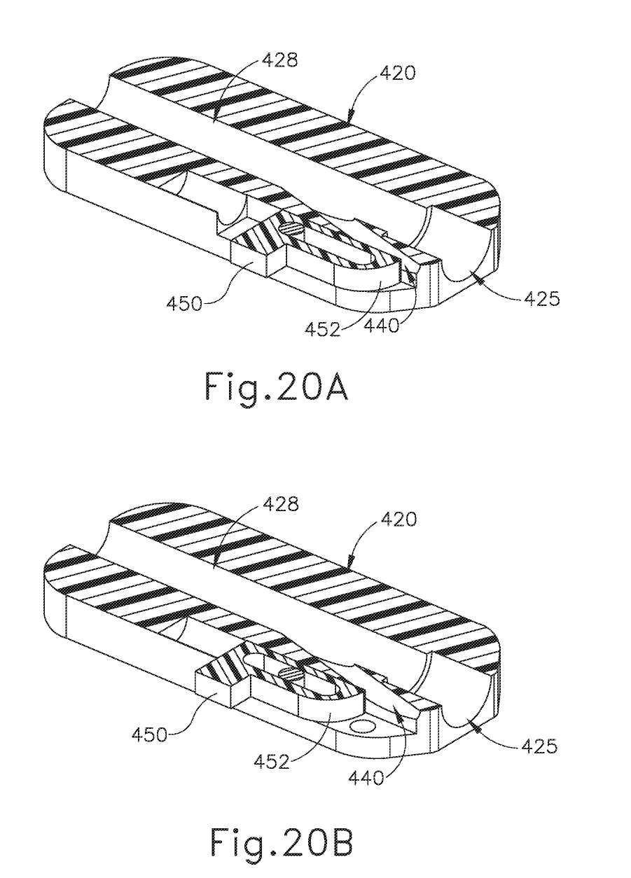

FIG. 17 shows an exemplary suction instrument assembly (400) that includes a suction instrument (410). As best seen in FIGS. 19-20B, suction instrument (410) of this example includes an integral navigation guidewire port (440). Suction instrument assembly (400) of this example further includes console (16), navigation guidewire (130), suction source (80), and conduit (90), each of which is described above. Suction instrument (410) of the present example includes an elongate cannula (412) extending distally from a grip portion (420). Cannula (412) has an open distal end (414) and a bent region (416) formed just distal to grip portion (420). Bent region (416) defines a bend angle that is selected to facilitate insertion of distal end (414) in a patient by an operator grasping grip portion (420). Various suitable bend angles that may be used will be apparent to those of ordinary skill in the art in view of the teachings herein.

In the present example, cannula (412) is rigid such that cannula (412) maintains the bend of bent region (416) and does not buckle during insertion into a patient's nasal cavity. By way of example only, cannula (412) may be formed of stainless steel (e.g., a stainless steel hypotube, etc.) and/or any other suitable rigid material. Also in the present example, cannula (412) defines a lumen with a diameter of approximately 2.44 mm. Alternatively, any other suitable diameter may be used. It should also be understood that the lumen of cannula (412) may have an elliptical cross-sectional profile or some other non-circular cross-sectional profile, if desired. A non-circular cross-sectional profile may provide additional clearance for other instruments to be positioned simultaneously in the same anatomical passageway (e.g., nasal cavity) with cannula (412).

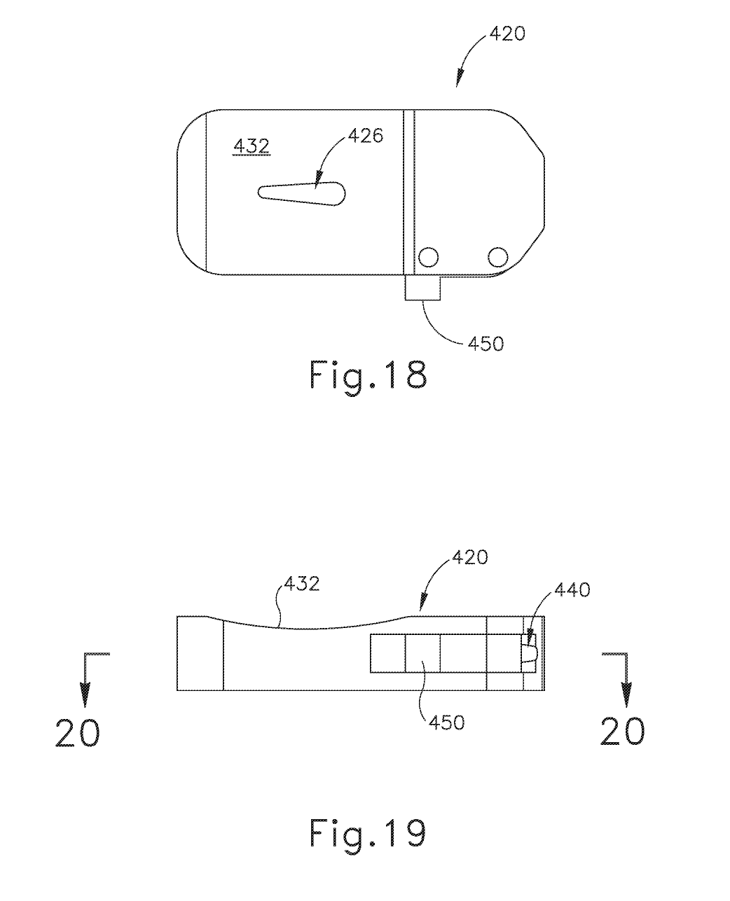

FIGS. 18-20B show grip portion (420) in greater detail. It should be understood that the rest of the components of suction instrument assembly (400) are omitted from FIGS. 18-20B, for clarity. Grip portion (420) of the present example includes a proximal suction conduit port (424) that is configured to couple with conduit (90). Port (424) is inserted in an opening (425) that is formed in grip portion (420), as seen in FIGS. 20A-20B. In the present example, conduit port (424) has a barbed configuration to promote a secure fit with an elastomeric conduit (90), though it should be understood that various other kinds of configurations may be used for port (224).

Grip portion (420) of the present example further includes a transverse vent opening (426) formed through an upper surface (432); and a lower surface (434). Vent opening (426) is in fluid communication with a lumen (428) formed through grip portion (420). Lumen (428) is best seen in FIGS. 20A-20B. As best seen in FIG. 18, vent opening (426) has a teardrop shape in the present example, though it should be understood that vent opening (426) may have any other suitable shape. By way of example only, the teardrop shape (or some other elongate shape) may enable the operator to selectively vary the amount of suction based on the longitudinal position of the operator's thumb (or other finger) on vent opening (426). Lumen (428) is further in fluid communication with suction port opening (425) and a lumen (not shown) of cannula (412). It should be understood that lumen (428) of grip portion (420) and the lumen of cannula (412) cooperate to provide an unobstructed fluid path from port (424) to open distal end (414) of cannula (412).

Surfaces (432, 434) are configured to promote gripping of grip portion (420) by an operator. In particular, as best seen in FIGS. 17 and 19, upper surface (432) provides a concave contour; while lower surface (434) is substantially flat. By way of example only, an operator may grasp grip portion (420) by placing a thumb on upper surface (432) and the side of the index finger of the same hand on lower surface (434). The generally rectangular shape of grip portion (420) may provide the operator with substantial purchase on grip portion (420), while the configurations of surfaces (432, 434) may further secure the operator's grip.

Grip portion (420) of the present example also includes navigation guidewire port (440), as noted above. As best seen in FIGS. 19-20B, navigation guidewire port (440) is in the form of a lumen that is in communication with lumen (428) of grip portion (420). Navigation guidewire (130) is slidably disposed in navigation guidewire port (440). Navigation guidewire (130) continues through lumen (428) of grip portion (420), and further through the lumen of cannula (412), such that the distal end of navigation guidewire (130) protrudes distally from distal end (414) of cannula (412). The proximal end of guidewire (130) is coupled with console (16).

Grip portion (420) of the present example further includes a locking member (450). Locking member (450) is secured to the body of grip portion (420) and is configured to translate longitudinally relative to the body of grip portion (420) through a limited range of motion. In particular, locking member (450) is configured to translate between a proximal position (FIG. 20A) and a distal position (FIG. 20B). While not shown, some variations of grip portion (420) include a coil spring or other resilient member that resiliently biases locking member (450) to the proximal position. When locking member (450) is in the proximal position, the proximal end of locking member (450) bears against the navigation guidewire (130) that is disposed in suction instrument (410), thereby providing a pinching effect against navigation guidewire (130). This pinching effect creates friction on navigation guidewire (130), thereby substantially securing the longitudinal position of navigation guidewire (130) relative to suction instrument (210). In other words, navigation guidewire (130) is in a locked state when locking member (450) is in the proximal position shown in FIG. 20A.

If the operator wishes to translate navigation guidewire (130) relative to suction instrument (410) (i.e., to advance or retract the distal end of navigation guidewire (130) relative to distal end (414) of cannula (412)), the operator may advance locking member (450) distally, against the bias of the coil spring or other resilient member (not shown). This eventually results in the configuration shown in FIG. 20B. In this state, the proximal end of locking member (450) no longer bears against navigation guidewire (130), thereby relieving the pinching effect on navigation guidewire (130). Navigation guidewire (130) is then capable of sliding freely relative to suction instrument (410), such that navigation guidewire (130) is in an unlocked state. Once the operator is satisfied with the adjusted longitudinal position of navigation guidewire (130) relative to suction instrument (410), the operator may release locking member (450). The coil spring or other resilient member (not shown) will then return locking member (450) back to the position shown in FIG. 20A, thereby locking the adjusted longitudinal position of navigation guidewire (130) relative to suction instrument (410). Locking member (450) and the body of grip portion (420) may include structural features that cooperate to restrict the movement of locking member (420) to the range of motion depicted between FIGS. 20A-20B.

In the present example, guidewire port (440) and locking member (450) are positioned such that locking member (450) is laterally offset from the vertical plane that passes through the entire length of cannula (412) and vent opening (426). Due to this positioning, an operator may tend to use a finger (other than the thumb) of the hand that grasps grip portion (420) to actuate locking member (450). The operator may this keep vent opening (426) covered with their thumb while actuating locking member (450). Of course, an operator may actuate locking member (450) in any other suitable fashion.

It should be understood that guidewire (130) of this example is configured and operable just like guidewire (30) described above. Thus, an operator may use IGS navigation system (1) to determine the location of the distal end of guidewire (130) within a patient, to thereby determine the location of distal end (414) of cannula (412) in the patient. Moreover, after transitioning locking member (450) to the unlocked position, the operator may freely slide guidewire (130) within lumen (428) of grip portion (420) and the lumen of cannula (412), to thereby facilitate probing with the distal end of guidewire (130). The diameters of lumen (428) and the lumen of cannula (412) are large enough to enable an effective amount of suction to reach distal end (414) even while guidewire (130) is disposed in lumen (428) of grip portion (420) and the lumen of cannula (412).

In the present example, and as best seen in FIGS. 20A-20B, navigation guidewire port (440) is obliquely oriented relative to the longitudinal axis of lumen (428) of grip portion (420); while opening (425) of suction conduit port (424) is coaxially aligned with the longitudinal axis of lumen (428) of grip portion (420). In some other versions, these relationships are reversed. In other words, navigation guidewire port (440) may be coaxially aligned with the longitudinal axis of lumen (428) of grip portion (420); while opening (425) of suction conduit port (424) is oriented obliquely relative to the longitudinal axis of lumen (428) of grip portion (420). Other suitable orientations and arrangements will be apparent to those of ordinary skill in the art in view of the teachings herein.

During use of suction instrument assembly (400), the operator may grasp grip portion (420) and position distal end (414) of cannula (412) at a target site in a patient. In some such instances, suction source (80) remains in a constantly activated state. In those instances, the operator may leave vent opening (426) uncovered as the operator positions instrument (410) relative to the patient. This may result in suction source (80) drawing suction through vent opening (426) without drawing suction through open distal end (414). During the act of positioning instrument (410), the operator may rely on navigation guidewire (130) and the rest of IGS navigation system (1) to provide image guidance as described above. This may include manipulation of locking member (450) to enable adjustment of the longitudinal position of navigation guidewire (130) relative to suction instrument (410).

When the operator reaches the target site and wishes to apply the suction to the target site in the patient via open distal end (414), the operator may simply cover vent opening (426) with the operator's thumb (or otherwise cover vent opening (426)). The operator may thus selectively cover and uncover vent opening during a procedure in order to selectively apply suction. Moreover, the operator may use navigation guidewire (130) and the rest of IGS navigation system (1) to provide image guidance to suction instrument (410) before, during, and/or after the suctioning procedure. This may include using locking member (450) to selectively lock and unlock the longitudinal position of navigation guidewire (130) relative to suction instrument (410).

In the present example, locking member (450) is configured to cooperate with the body of grip portion (420) and navigation guidewire (130) to substantially seal guidewire port (440) when locking member (450) is in the locked position shown in FIG. 20A. Thus, when navigation guidewire (130) and locking member (450) are in a locked state, suction instrument (410) will not lose meaningful suction through guidewire port (440). Moreover, locking member (450) will prevent loss of suction through guidewire port (440) when a navigation guidewire (130) is not disposed in guidewire port (440). Other features that may be used to prevent loss of suction will be apparent to those of ordinary skill in the art in view of the teachings herein.

C. Third Exemplary Guidewire Locking Feature

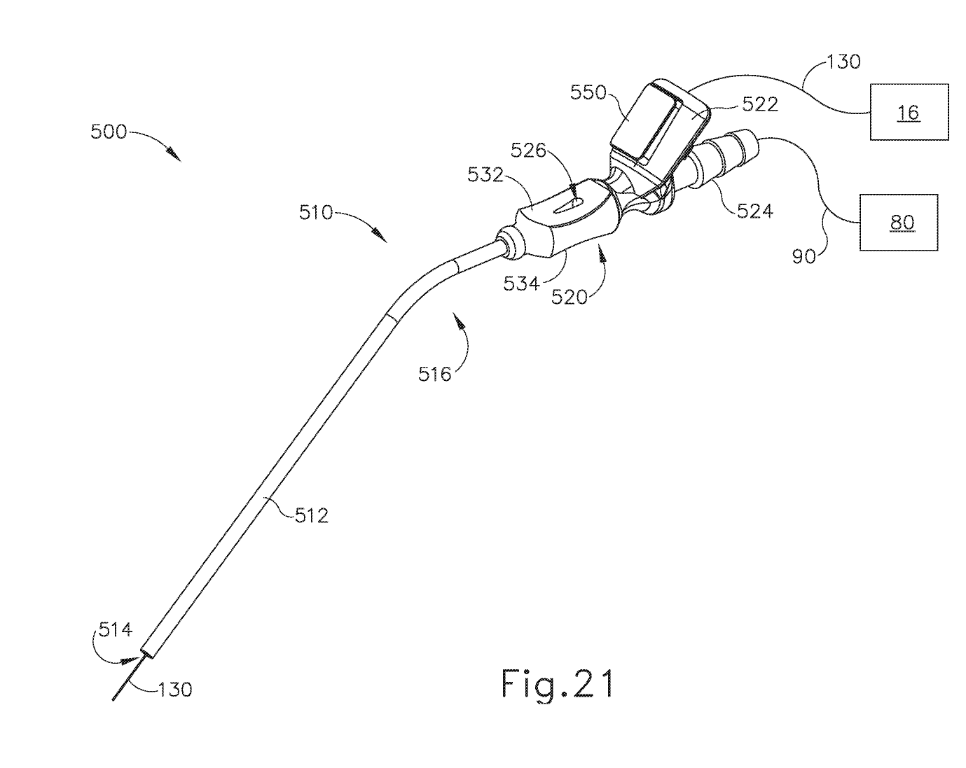

FIG. 21 shows an exemplary suction instrument assembly (500) that includes a suction instrument (510) having an integral navigation guidewire port (522). Suction instrument assembly (500) of this example further includes console (16), navigation guidewire (130), suction source (80), and conduit (90), each of which is described above. Suction instrument (510) of the present example includes an elongate cannula (512) extending distally from a grip portion (520). Cannula (512) has an open distal end (514) and a bent region (516) formed just distal to grip portion (520). Bent region (516) defines a bend angle that is selected to facilitate insertion of distal end (514) in a patient by an operator grasping grip portion (520). Various suitable bend angles that may be used will be apparent to those of ordinary skill in the art in view of the teachings herein.

In the present example, cannula (512) is rigid such that cannula (512) maintains the bend of bent region (516) and does not buckle during insertion into a patient's nasal cavity. By way of example only, cannula (512) may be formed of stainless steel (e.g., a stainless steel hypotube, etc.) and/or any other suitable rigid material. Also in the present example, cannula (512) defines a lumen with a diameter of approximately 2.44 mm. Alternatively, any other suitable diameter may be used. It should also be understood that the lumen of cannula (512) may have an elliptical cross-sectional profile or some other non-circular cross-sectional profile, if desired. A non-circular cross-sectional profile may provide additional clearance for other instruments to be positioned simultaneously in the same anatomical passageway (e.g., nasal cavity) with cannula (512).

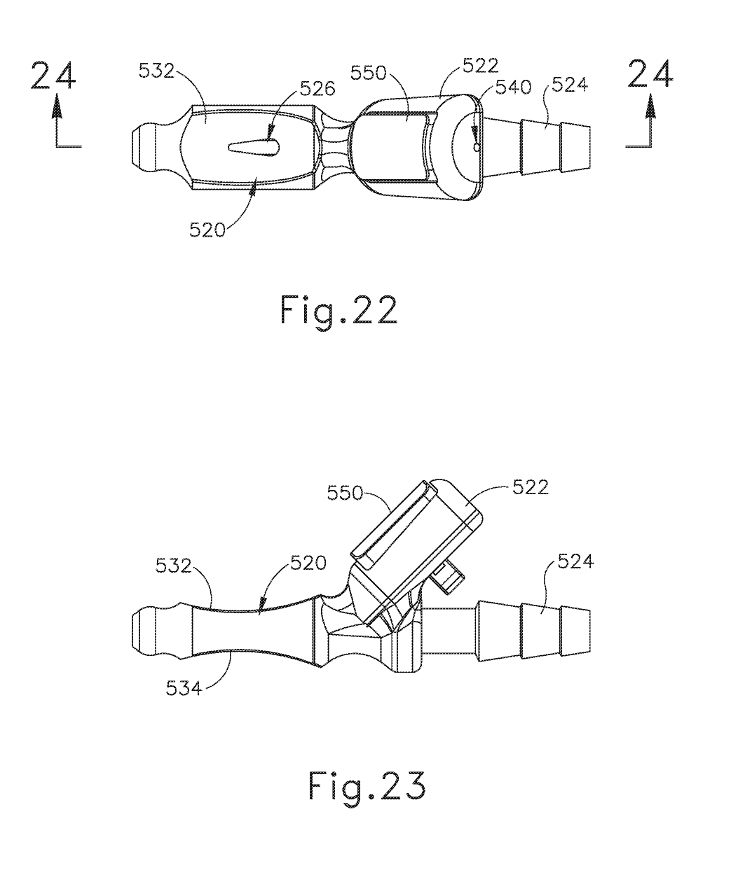

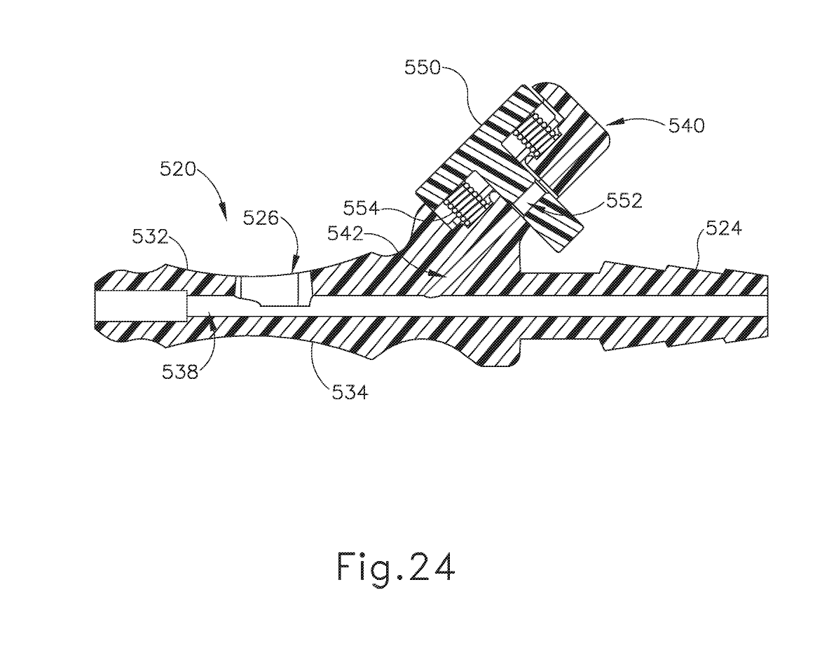

FIGS. 22-25B show grip portion (520) in greater detail. It should be understood that the rest of the components of suction instrument assembly (500) are omitted from FIGS. 22-25B, for clarity. Grip portion (520) of the present example includes a proximal suction conduit port (524) that is configured to couple with conduit (90). In the present example, port (524) has a barbed configuration to promote a secure fit with an elastomeric conduit (90), though it should be understood that various other kinds of configurations may be used for port (524). Grip portion (520) of the present example further includes a transverse vent opening (526) formed through an upper surface (532); and a lower surface (534). As best seen in FIG. 24, vent opening (526) is in fluid communication with a lumen (538) formed through grip portion (520). As best seen in FIG. 22, vent opening (526) has a teardrop shape in the present example, though it should be understood that vent opening (526) may have any other suitable shape. By way of example only, the teardrop shape (or some other elongate shape) may enable the operator to selectively vary the amount of suction based on the longitudinal position of the operator's thumb (or other finger) on vent opening (526). Lumen (538) is further in fluid communication with suction conduit port (524) and a lumen (not shown) of cannula (512). It should be understood that lumen (538) of grip portion (520) and the lumen of cannula (512) cooperate to provide an unobstructed fluid path from port (524) to open distal end (514) of cannula (512).

Surfaces (532, 534) are configured to promote gripping of grip portion (520) by an operator. In particular, as best seen in FIGS. 23-25B, each surface (532, 534) provides a respective concave contour. By way of example only, an operator may grasp grip portion (520) by placing a thumb on upper surface (532) and the side of the index finger of the same hand on lower surface (534). The generally rectangular shape of grip portion (520) may provide the operator with substantial purchase on grip portion (520), while the configurations of surfaces (532, 534) may further secure the operator's grip.

Grip portion (520) of the present example also includes navigation guidewire port (522), as noted above. As best seen in FIGS. 22-24, navigation guidewire port (522) is in the form of an arm that defines an opening (540) leading to a lumen (542). In some versions, the outer region of opening (540) is funnel-shaped or tapered to facilitate insertion of navigation guidewire (130) into opening (540) and lumen (542). Lumen (542) of navigation guidewire port (522) is in communication with lumen (538) of grip portion (520). Navigation guidewire (130) is slidably disposed in navigation guidewire port (522). Navigation guidewire (130) continues through lumen (538) of grip portion (520), and further through the lumen of cannula (512), such that the distal end of navigation guidewire (130) protrudes distally from distal end (514) of cannula (512). The proximal end of guidewire (130) is coupled with console (16).

Grip portion (520) of the present example further includes a locking member (550), which is in the form of a push-button in the present example. Locking member (550) is secured to guidewire port (522) and is configured to translate transversely relative to guidewire port (522) through a limited range of motion. Locking member (550) defines a lumen (552) that is configured to selectively align with lumen (542) of guidewire port (522). A coil spring (554) is configured to urge locking member (550) toward the position shown in FIG. 24, where lumen (552) is not aligned with lumen (542). While a coil spring (554) is used to provide a resilient bias in the present example, it should be understood that any other suitable kind of resilient member may be used.

In the present example, guidewire port (522) and locking member (550) are positioned such that locking member (550) is aligned with the vertical plane that passes through the entire length of cannula (512) and vent opening (526). Due to this positioning, an operator may tend to use the thumb of the hand that grasps grip portion (520) to actuate locking member (550). This may be the same thumb that selectively covers and uncovers vent opening (526). The operator may thus transition between keeping vent opening (526) covered with their thumb and actuating locking member (550). Of course, an operator may actuate locking member (550) in any other suitable fashion.

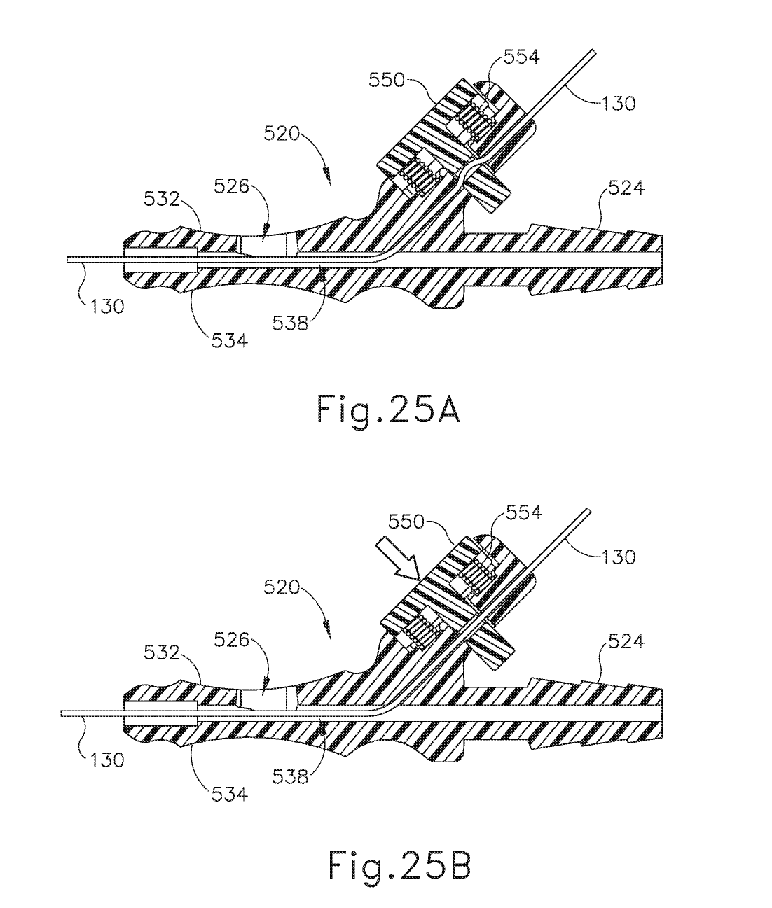

FIGS. 25A-25B show the relationship between locking member (550) and navigation guidewire (130). In FIG. 25A, locking member (550) is in an outward position to provide a locked state. In this position, lumen (552) of locking member (550) is not aligned with lumen (542) of guidewire port (522). Since navigation guidewire (130) is disposed in both lumens (542, 552), the non-alignment of lumens (542, 552) provides a pinching effect on navigation guidewire (130). This pinching effect creates friction on navigation guidewire (130), thereby substantially securing the longitudinal position of navigation guidewire (130) relative to suction instrument (510). In other words, navigation guidewire (130) is in a locked state in FIG. 25A.

If the operator wishes to translate navigation guidewire (130) relative to suction instrument (510) (i.e., to advance or retract the distal end of navigation guidewire (130) relative to distal end (514) of cannula (512)), the operator may press locking member (550) inwardly, against the bias of coil spring (554). This eventually results in the configuration shown in FIG. 25B. In this state, lumens (542, 552) are aligned with each other, thereby relieving the pinching effect on navigation guidewire (130). Navigation guidewire (130) is then capable of sliding freely relative to suction instrument (510), such that navigation guidewire (130) is in an unlocked state. Once the operator is satisfied with the adjusted longitudinal position of navigation guidewire (130) relative to suction instrument (510), the operator may release locking member (550). Coil spring (554) will then return locking member (550) back to the position shown in FIG. 25A, thereby locking the adjusted longitudinal position of navigation guidewire (130) relative to suction instrument (510). Locking member (550) and guidewire port (522) may include structural features that cooperate to restrict the movement of locking member (550) to the range of motion depicted between FIGS. 25A-25B.

It should be understood that guidewire (130) of this example is configured and operable just like guidewire (30) described above. Thus, an operator may use IGS navigation system (1) to determine the location of the distal end of guidewire (130) within a patient, to thereby determine the location of distal end (514) of cannula (512) in the patient. Moreover, after transitioning locking member (550) to the unlocked position, the operator may freely slide guidewire (130) within lumen (538) of grip portion (520) and the lumen of cannula (512), to thereby facilitate probing with the distal end of guidewire (130). The diameters of lumen (538) and the lumen of cannula (512) are large enough to enable an effective amount of suction to reach distal end (514) even while guidewire (130) is disposed in lumen (538) of grip portion (520) and the lumen of cannula (512).

In the present example, and as best seen in FIGS. 23-25B, navigation guidewire port (522) is obliquely oriented relative to the longitudinal axis of lumen (538) of grip portion (520); while conduit port (524) is coaxially aligned with the longitudinal axis of lumen (538) of grip portion (520). In some other versions, these relationships are reversed. In other words, navigation guidewire port (522) may be coaxially aligned with the longitudinal axis of lumen (538) of grip portion (520); while conduit port (524) is oriented obliquely relative to the longitudinal axis of lumen (538) of grip portion (520). Other suitable orientations and arrangements will be apparent to those of ordinary skill in the art in view of the teachings herein.

During use of suction instrument assembly (500), the operator may grasp grip portion (520) and position distal end (514) of cannula (512) at a target site in a patient. In some such instances, suction source (80) remains in a constantly activated state. In those instances, the operator may leave vent opening (526) uncovered as the operator positions instrument (510) relative to the patient. This may result in suction source (80) drawing suction through vent opening (526) without drawing suction through open distal end (514). During the act of positioning instrument (510), the operator may rely on navigation guidewire (130) and the rest of IGS navigation system (1) to provide image guidance as described above. This may include manipulation of locking member (550) to enable adjustment of the longitudinal position of navigation guidewire (130) relative to suction instrument (510).

When the operator reaches the target site and wishes to apply the suction to the target site in the patient via open distal end (514), the operator may simply cover vent opening (526) with the operator's thumb (or otherwise cover vent opening (526)). The operator may thus selectively cover and uncover vent opening during a procedure in order to selectively apply suction. Moreover, the operator may use navigation guidewire (130) and the rest of IGS navigation system (1) to provide image guidance to suction instrument (510) before, during, and/or after the suctioning procedure. This may include using locking member (550) to selectively lock and unlock the longitudinal position of navigation guidewire (130) relative to suction instrument (510).

In the present example, locking member (550) is configured to cooperate with guidewire port (540) and navigation guidewire (130) to substantially seal guidewire port (540) when locking member (550) is in the locked position shown in FIG. 25A. Thus, when navigation guidewire (130) and locking member (550) are in a locked state, suction instrument (510) will not lose meaningful suction through guidewire port (540). Moreover, locking member (550) will prevent loss of suction through guidewire port (540) when a navigation guidewire (130) is not disposed in guidewire port (540). Other features that may be used to prevent loss of suction will be apparent to those of ordinary skill in the art in view of the teachings herein.

D. Fourth Exemplary Guidewire Locking Feature

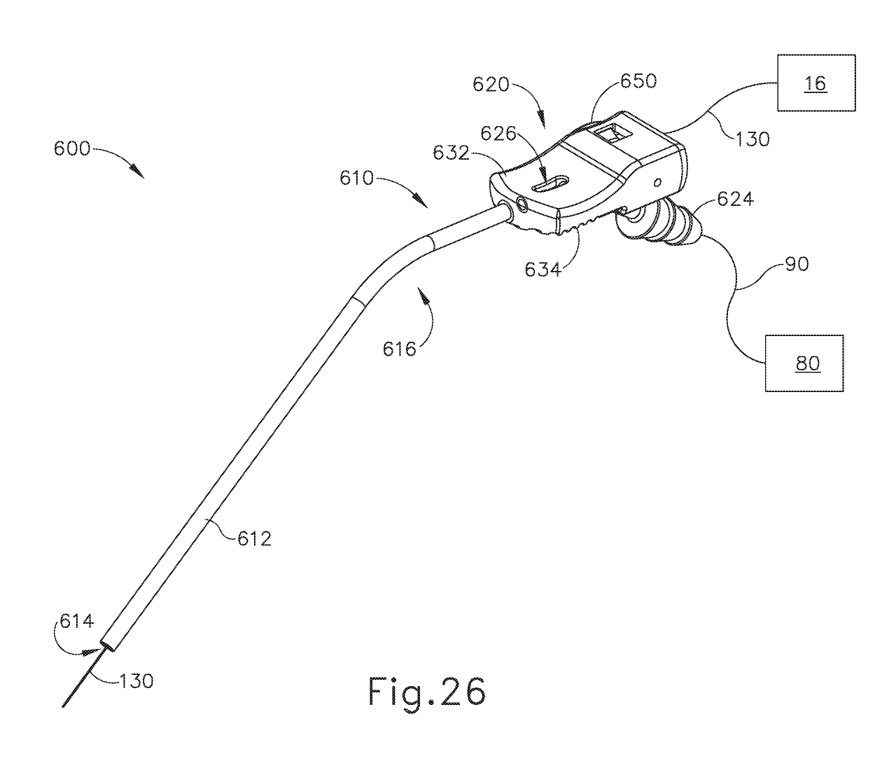

FIG. 26 shows an exemplary suction instrument assembly (600) that includes a suction instrument (610) having an integral navigation guidewire port (640). Suction instrument assembly (600) of this example further includes console (16), navigation guidewire (130), suction source (80), and conduit (90), each of which is described above. Suction instrument (610) of the present example includes an elongate cannula (612) extending distally from a grip portion (620). Cannula (612) has an open distal end (614) and a bent region (616) formed just distal to grip portion (620). Bent region (616) defines a bend angle that is selected to facilitate insertion of distal end (614) in a patient by an operator grasping grip portion (620). Various suitable bend angles that may be used will be apparent to those of ordinary skill in the art in view of the teachings herein.

In the present example, cannula (612) is rigid such that cannula (612) maintains the bend of bent region (616) and does not buckle during insertion into a patient's nasal cavity. By way of example only, cannula (612) may be formed of stainless steel (e.g., a stainless steel hypotube, etc.) and/or any other suitable rigid material. Also in the present example, cannula (612) defines a lumen with a diameter of approximately 2.44 mm. Alternatively, any other suitable diameter may be used. It should also be understood that the lumen of cannula (612) may have an elliptical cross-sectional profile or some other non-circular cross-sectional profile, if desired. A non-circular cross-sectional profile may provide additional clearance for other instruments to be positioned simultaneously in the same anatomical passageway (e.g., nasal cavity) with cannula (612).

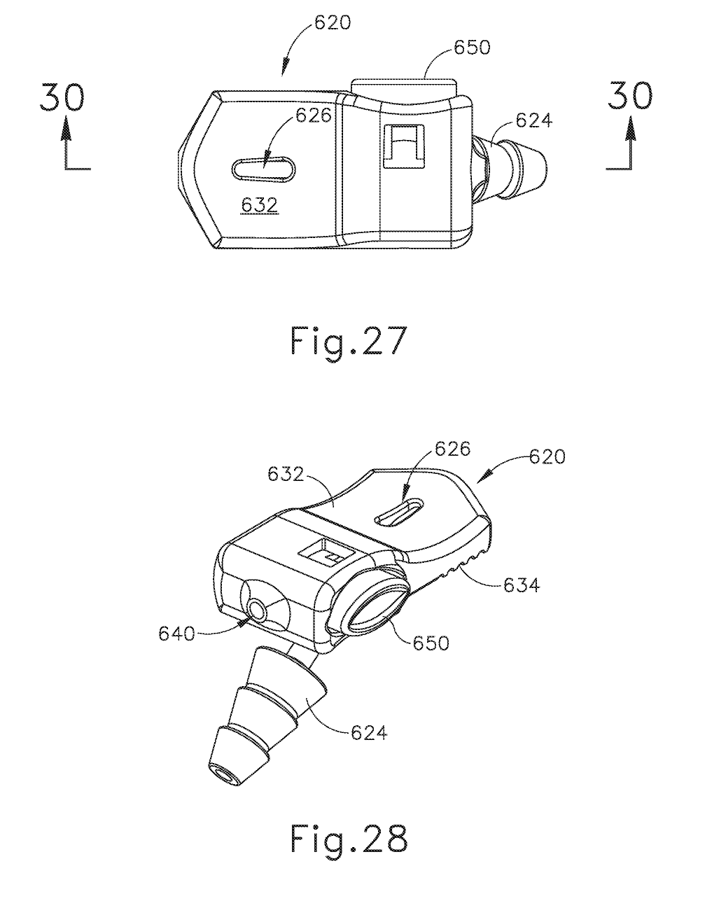

FIGS. 27-31B show grip portion (620) in greater detail. It should be understood that the rest of the components of suction instrument assembly (600) are omitted from FIGS. 27-31B, for clarity. Grip portion (620) of the present example includes a proximal suction conduit port (624) that is configured to couple with conduit (90). In the present example, port (624) has a barbed configuration to promote a secure fit with an elastomeric conduit (90), though it should be understood that various other kinds of configurations may be used for port (624). Grip portion (620) of the present example further includes a transverse vent opening (626) formed through an upper surface (632); and a lower surface (634). As best seen in FIG. 30, vent opening (626) is in fluid communication with a lumen (628) formed through grip portion (620). As best seen in FIG. 27, vent opening (626) has a teardrop shape in the present example, though it should be understood that vent opening (626) may have any other suitable shape. By way of example only, the teardrop shape (or some other elongate shape) may enable the operator to selectively vary the amount of suction based on the longitudinal position of the operator's thumb (or other finger) on vent opening (626). Lumen (628) is further in fluid communication with suction conduit port (624) and a lumen (not shown) of cannula (612). It should be understood that lumen (638) of grip portion (620) and the lumen of cannula (612) cooperate to provide an unobstructed fluid path from port (624) to open distal end (614) of cannula (612).

Surfaces (632, 634) are configured to promote gripping of grip portion (620) by an operator. In particular, as best seen in FIGS. 28 and 30, upper surface (632) provides a concave contour; while lower surface (634) provides a set of ridges. By way of example only, an operator may grasp grip portion (620) by placing a thumb on upper surface (632) and the side of the index finger of the same hand on lower surface (634). The generally rectangular shape of grip portion (620) may provide the operator with substantial purchase on grip portion (620), while the configurations of surfaces (632, 634) may further secure the operator's grip.

Grip portion (620) of the present example also includes navigation guidewire port (640), as noted above. As best seen in FIGS. 28-30, navigation guidewire port (640) is in the form of an opening that is coaxially aligned with lumen (628). In the present example, the outer region of navigation guidewire port (640) is funnel-shaped or tapered to facilitate insertion of navigation guidewire (130) into navigation guidewire port (640). Navigation guidewire (130) is slidably disposed in navigation guidewire port (640). Navigation guidewire (130) continues through lumen (628) of grip portion (620), and further through the lumen of cannula (612), such that the distal end of navigation guidewire (130) protrudes distally from distal end (614) of cannula (612). The proximal end of guidewire (130) is coupled with console (16).

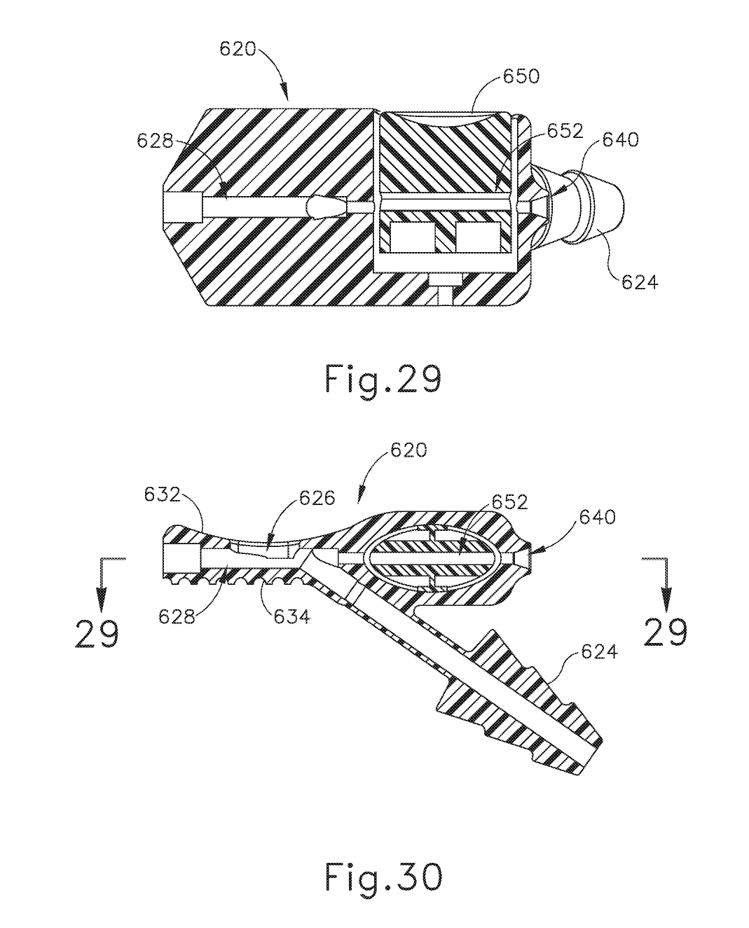

Grip portion (620) of the present example further includes a locking member (650), which is in the form of a push-button in the present example. Locking member (650) is secured to the body of grip portion (620) and is configured to translate transversely relative to guidewire port (640) through a limited range of motion. As best seen in FIG. 29, locking member (650) defines a lumen (652) that is configured to selectively align with guidewire port (640) and lumen (628) of grip portion (620). While not shown, some variations of grip portion (620) include a coil spring or other resilient member that resiliently biases locking member (550) to the outer position shown in FIG. 29.

In the present example, guidewire port (640) and locking member (650) are positioned such that locking member (650) is laterally offset from the vertical plane that passes through the entire length of cannula (612) and vent opening (626). Due to this positioning, an operator may tend to use a finger (other than the thumb) of the hand that grasps grip portion (620) to actuate locking member (650). The operator may this keep vent opening (626) covered with their thumb while actuating locking member (650). Of course, an operator may actuate locking member (650) in any other suitable fashion.

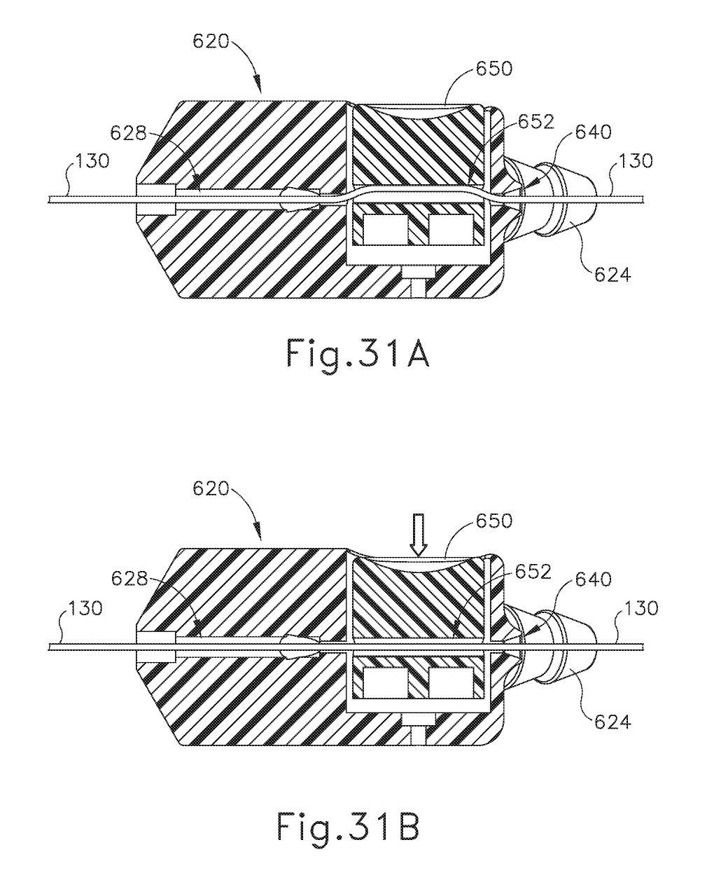

FIGS. 31A-31B show the relationship between locking member (650) and navigation guidewire (130). In FIG. 31A, locking member (650) is in an outward position to provide a locked state. In this position, lumen (652) of locking member (550) is not aligned with guidewire port (640) or lumen (628) of grip portion (620). Since navigation guidewire (130) is disposed in guidewire port (640), lumen (652), and lumen (628) of grip portion (620), the non-alignment of port (640) and lumens (628, 654) provides a pinching effect on navigation guidewire (130). This pinching effect creates friction on navigation guidewire (130), thereby substantially securing the longitudinal position of navigation guidewire (130) relative to suction instrument (610). In other words, navigation guidewire (130) is in a locked state in FIG. 31A.

If the operator wishes to translate navigation guidewire (130) relative to suction instrument (610) (i.e., to advance or retract the distal end of navigation guidewire (130) relative to distal end (614) of cannula (612)), the operator may press locking member (650) inwardly, against the bias of the coil spring or other resilient member that biases locking member (650). This eventually results in the configuration shown in FIG. 31B. In this state, port (640) and lumens (628, 654) are aligned with each other, thereby relieving the pinching effect on navigation guidewire (130). Navigation guidewire (130) is then capable of sliding freely relative to suction instrument (610), such that navigation guidewire (130) is in an unlocked state. Once the operator is satisfied with the adjusted longitudinal position of navigation guidewire (130) relative to suction instrument (610), the operator may release locking member (650). The coil spring or other resilient member that biases locking member (650) will then return locking member (650) back to the position shown in FIG. 31A, thereby locking the adjusted longitudinal position of navigation guidewire (130) relative to suction instrument (610). Locking member (650) and the body of grip portion (620) may include structural features that cooperate to restrict the movement of locking member (650) to the range of motion depicted between FIGS. 31A-31B.

It should be understood that guidewire (130) of this example is configured and operable just like guidewire (30) described above. Thus, an operator may use IGS navigation system (1) to determine the location of the distal end of guidewire (130) within a patient, to thereby determine the location of distal end (614) of cannula (612) in the patient. Moreover, after transitioning locking member (650) to the unlocked position, the operator may freely slide guidewire (130) within lumen (628) of grip portion (620) and the lumen of cannula (612), to thereby facilitate probing with the distal end of guidewire (130). The diameters of lumen (628) and the lumen of cannula (612) are large enough to enable an effective amount of suction to reach distal end (614) even while guidewire (130) is disposed in lumen (628) of grip portion (620) and the lumen of cannula (512).

In the present example, and as best seen in FIGS. 26-31B, navigation guidewire port (640) is coaxially aligned with the longitudinal axis of lumen (628) of grip portion (620); while conduit port (624) is oriented obliquely relative to the longitudinal axis of lumen (628) of grip portion (620). In some other versions, these relationships are reversed. In other words, navigation guidewire port (640) may be obliquely oriented relative to the longitudinal axis of lumen (628) of grip portion (620); while conduit port (624) is coaxially aligned with the longitudinal axis of lumen (638) of grip portion (620). Other suitable orientations and arrangements will be apparent to those of ordinary skill in the art in view of the teachings herein.

During use of suction instrument assembly (600), the operator may grasp grip portion (620) and position distal end (614) of cannula (612) at a target site in a patient. In some such instances, suction source (80) remains in a constantly activated state. In those instances, the operator may leave vent opening (626) uncovered as the operator positions instrument (610) relative to the patient. This may result in suction source (80) drawing suction through vent opening (626) without drawing suction through open distal end (614). During the act of positioning instrument (610), the operator may rely on navigation guidewire (130) and the rest of IGS navigation system (1) to provide image guidance as described above. This may include manipulation of locking member (650) to enable adjustment of the longitudinal position of navigation guidewire (130) relative to suction instrument (610).

When the operator reaches the target site and wishes to apply the suction to the target site in the patient via open distal end (614), the operator may simply cover vent opening (626) with the operator's thumb (or otherwise cover vent opening (626)). The operator may thus selectively cover and uncover vent opening during a procedure in order to selectively apply suction. Moreover, the operator may use navigation guidewire (130) and the rest of IGS navigation system (1) to provide image guidance to suction instrument (610) before, during, and/or after the suctioning procedure. This may include using locking member (650) to selectively lock and unlock the longitudinal position of navigation guidewire (130) relative to suction instrument (610).

In the present example, locking member (650) is configured to cooperate with guidewire port (640) and navigation guidewire (130) to substantially seal guidewire port (640) when locking member (650) is in the locked position shown in FIG. 31A. Thus, when navigation guidewire (130) and locking member (650) are in a locked state, suction instrument (610) will not lose meaningful suction through guidewire port (640). Moreover, locking member (650) will prevent loss of suction through guidewire port (640) when a navigation guidewire (130) is not disposed in guidewire port (640). Other features that may be used to prevent loss of suction will be apparent to those of ordinary skill in the art in view of the teachings herein.

VI. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An apparatus comprising: (a) a cannula including: (i) a proximal end, (ii) a distal end, and (iii) a first lumen extending from the proximal end to the distal end, wherein the cannula is formed of a rigid material; and (b) a grip portion secured to the proximal end of the cannula, wherein the grip portion includes: (i) a second lumen, wherein the second lumen is in communication with the first lumen, (ii) a transversely oriented vent port, wherein the transversely oriented vent port is configured to communicate with the second lumen to thereby vent the second lumen to atmosphere, (iii) a suction port, wherein the suction port is configured to communicate with the second lumen to thereby communicate suction to the distal end of the cannula via the first lumen, and (iv) a guidewire port, wherein the guidewire port is configured to receive a guidewire, wherein the guidewire port is further configured to communicate with the second lumen to thereby provide a path for the guidewire to reach the first lumen.

### EXAMPLE 2

The apparatus of Example 1, wherein the cannula further includes a preformed bend formed between the proximal end and the distal end.

### EXAMPLE 3

The apparatus of any one or more of Examples 1 through 2, wherein the cannula is formed of rigid steel.

### EXAMPLE 4

The apparatus of any one or more of Examples 1 through 3, wherein the second lumen defines a longitudinal axis, wherein the suction port is oriented obliquely relative to the longitudinal axis.

### EXAMPLE 5

The apparatus of Example 4, wherein the guidewire port is coaxially aligned with the longitudinal axis.

### EXAMPLE 6

The apparatus of any one or more of Examples 1 through 3, wherein the second lumen defines a longitudinal axis, wherein the guidewire port is oriented obliquely relative to the longitudinal axis.

### EXAMPLE 7

The apparatus of Example 6, wherein the suction port is coaxially aligned with the longitudinal axis.

### EXAMPLE 8

The apparatus of any one or more of Examples 1 through 3, wherein the second lumen defines a longitudinal axis, wherein the guidewire port is oriented obliquely relative to the longitudinal axis, wherein the suction port is oriented obliquely relative to the longitudinal axis.

### EXAMPLE 9

The apparatus of any one or more of Examples 1 through 8, wherein the guidewire port defines a third lumen, wherein the third lumen distally terminates at the second lumen.

### EXAMPLE 10

The apparatus of Example 9, wherein the vent port is located at a first longitudinal position along the second lumen, wherein the third lumen distally terminates at the second lumen at a second longitudinal position along the second lumen, wherein the second longitudinal position is distal to the first longitudinal position.

### EXAMPLE 11

The apparatus of any one or more of Examples 1 through 10, wherein the grip portion further includes a locking feature, wherein the locking feature is configured to selectively engage a guidewire disposed in the guidewire port to thereby selectively secure the position of the guidewire relative to the grip portion by moving between a locked position and an unlocked position.

### EXAMPLE 12

The apparatus of Example 11, wherein the grip portion further includes: (i) a body, wherein the vent port is formed in the body, and (ii) an arm extending obliquely from the body, wherein the guidewire port is located on the arm, wherein the locking feature is located on the arm.

### EXAMPLE 13

The apparatus of Example 12, wherein the locking feature comprises a button, wherein the button is configured to translate transversely relative to a longitudinal axis defined by the arm to thereby transition between the locked position and the unlocked position.

### EXAMPLE 14

The apparatus of any one or more of Examples 12 through 13, wherein the cannula, the vent port, and the arm are aligned along a shared plane.

### EXAMPLE 15

The apparatus of any one or more of Examples 12 through 13, wherein the cannula and the vent port are aligned along a first plane, wherein the arm is aligned along a second plane, wherein the second plane is transverse to the first plane.

### EXAMPLE 16

The apparatus of any one or more of Examples 11 through 15, wherein the locking feature is resiliently biased toward the locked position.

### EXAMPLE 17

The apparatus of one or more of Examples 11 through 16, wherein the locking feature is further configured to provide a fluid seal of the guidewire port when the locking feature is in the locked position.

### EXAMPLE 18

The apparatus of one or more of Examples 11 through 17, wherein the guidewire port defines a third lumen, wherein the locking feature defines a fourth lumen, wherein the third and fourth lumens are configured to receive the guidewire, wherein the locking feature in the unlocked position is configured to provide alignment of the third and fourth lumens, wherein the locking feature in the locked position is configured to provide non-alignment of the third and fourth lumens.

### EXAMPLE 19

An apparatus comprising: (a) a cannula including: (i) a proximal end, (ii) a distal end, and (iii) a first lumen extending from the proximal end to the distal end, wherein the cannula is formed of a rigid material; and (b) a grip portion secured to the proximal end of the cannula, wherein the grip portion includes: (i) a second lumen, wherein the second lumen is in communication with the first lumen, (ii) a suction port, wherein the suction port is configured to communicate with the second lumen to thereby communicate suction to the distal end of the cannula via the first lumen, (iii) a guidewire port, wherein the guidewire port is configured to receive a guidewire, wherein the guidewire port is further configured to communicate with the second lumen to thereby provide a path for the guidewire to reach the first lumen, and (iv) a locking feature, wherein the locking feature is configured to selectively engage a guidewire disposed in the guidewire port to thereby selectively secure the position of the guidewire relative to the grip portion.

### EXAMPLE 20

An apparatus comprising: (a) a cannula including: (i) a proximal end, (ii) a distal end, and (iii) a first lumen extending from the proximal end to the distal end; (b) a grip portion secured to the proximal end of the cannula, wherein the grip portion includes: (i) a second lumen, wherein the second lumen is in communication with the first lumen, (ii) a suction port, wherein the suction port is configured to communicate with the second lumen to thereby communicate suction to the distal end of the cannula via the first lumen, and (iii) a guidewire port, wherein the guidewire port is configured to receive a guidewire, wherein the guidewire port is further configured to communicate with the second lumen to thereby provide a path for the guidewire to reach the first lumen; and (c) a navigation guidewire configured to fit in the guidewire port, wherein the navigation guidewire is further configured to fit in the first lumen, wherein the navigation guidewire includes a sensor, wherein the sensor is configured to generate data based on a location of the sensor within a patient; wherein the first lumen is configured to provide suction through the distal end while the navigation guidewire is disposed in the first lumen.

### EXAMPLE 21

A method of applying suction in a patient, the method comprising: (a) inserting a cannula into a patient, wherein the cannula defines a lumen; (b) identifying a location of the cannula in the patient based on image feedback from a navigation system, wherein the navigation system includes a guidewire with a sensor, wherein the guidewire is disposed in the lumen of the cannula; (c) positioning a distal end of the cannula at a target site in the patient based on image feedback from the navigation system; and (d) applying suction at the target site via the lumen and the distal end of the cannula.

### EXAMPLE 22

The method of Example 21, wherein the act of inserting the cannula into the patient comprises inserting the cannula into a nasal cavity of the patient.

### EXAMPLE 23

The method of any one or more of Examples 21 through 22, wherein the guidewire is disposed in the lumen of the cannula during the act of inserting the cannula into the patient.

### EXAMPLE 24

The method of any one or more of Examples 21 through 23, further comprising inserting the guidewire into the lumen of the cannula.

### EXAMPLE 25

The method of any one or more of Examples 21 through 24, wherein the guidewire has a distal end located distal to the distal end of the cannula during the act of positioning the distal end of the cannula at the target site.

### EXAMPLE 26

The method of any one or more of Examples 21 through 25, wherein the navigation system generates an electromagnetic field around a portion of the patient associated with the target site.

### EXAMPLE 27

The method of Example 26, wherein the sensor provides a variable signal based on the location of the sensor within the electromagnetic field.

### EXAMPLE 28

The method of any one or more of Examples 21 through 27, further comprising grasping a grip portion located at a proximal end of the cannula, wherein the grip portion has a vent opening.

### EXAMPLE 29

The method of Example 28, wherein the act of applying suction comprises covering the vent opening.

### EXAMPLE 30

The method of any one or more of Examples 28 through 29, wherein the vent opening is uncovered during the acts of inserting the cannula of the patient and positioning the distal end of the cannula at the target site.

### EXAMPLE 31

The method of any one or more of Examples 21 through 30, further comprising selectively locking a longitudinal position of the guidewire relative to the cannula.

### EXAMPLE 32

The method of Example 31, wherein the act of selectively locking comprises releasing an actuator.

### EXAMPLE 33

The method of Example 32, further comprising selectively unlocking the longitudinal position of the guidewire relative to the cannula.

### EXAMPLE 34

The method of Example 33, wherein the act of selectively unlocking comprises actuating the actuator.

### EXAMPLE 35

The method of Example 34, wherein the act of actuating the actuator comprise pressing the actuator in a direction transverse to a longitudinal axis of the guidewire.

### EXAMPLE 36

The method of any one or more of Examples 31 through 35, wherein the act of selectively locking comprises pinching a portion of the guidewire.

VII. Miscellaneous

The examples in the detailed description above include the use of navigation guidewires (130) that are configured for use with an IGS navigation system (1). However, it should be understood that the same instruments (60, 210, 310, 410, 510, 610) may also be used with any other suitable conventional guidewire. By way of example only, the above teachings may be readily applied in contexts where navigation guidewire (130) is substituted with a guidewire having one or more optical fibers that are operable to transmit light to the distal end of the guidewire. Such an illuminating guidewire may be provided in accordance with the teachings of U.S. Pat. No. 9,155,492, entitled “Sinus Illumination Lightwire Device,” issued Oct. 13, 2015, the disclosure of which is incorporated by reference herein. An example of such an illuminating guidewire is the Relieva Luma Sentry™ Sinus Illumination System by Acclarent, Inc. of Irvine, Calif. Other suitable guidewires that may be used with instruments (60, 210, 310, 410, 510, 610) in accordance with the teachings herein will be apparent to those of ordinary skill in the art.

In addition to having any of the foregoing features and functionalities, the various examples described herein may further incorporate one or more features and functionalities of the devices disclosed in U.S. patent application Ser. No. 15/861,959, entitled “Navigation Guidewire with Interlocked Coils,” filed on Jan. 4, 2018, the disclosure of which is incorporated by reference herein. Various suitable ways in which the teachings herein may be combined with the teachings of U.S. patent application Ser. No. 15/861,959 will be apparent to those of ordinary skill in the art.

The examples described above include the incorporation of a navigation coil or other navigation sensor in the distal end of guidewire (30, 130) to enable navigation and guidance via IGS system (1). In addition to, or in lieu of, providing such a navigation coil or other navigation sensor in the distal end of guidewire (30, 130), some versions may also incorporate one or more navigation coils or other navigation sensors in one or more other locations. By way of example only, one or more other navigation coils or other navigation sensors in grip portions (70, 220, 320, 420, 520, 620) and/or in some other component that will remain external to the patient during use of the device. Various other suitable locations where one or more other navigation coils or other navigation sensors may be provided will be apparent to those of ordinary skill in the art in view of the teachings herein.

Any of the devices herein may be modified and/or used in accordance with at least some of the teachings of U.S. Pub. No. 2016/0310042, entitled “System and Method to Map Structures of Nasal Cavity,” published Oct. 27, 2016, the disclosure of which is incorporated by reference herein. Thus, the devices herein may be used to provide mapping of anatomy within and adjacent to a patient's nasal cavity. Similarly, the devices herein may be used to provide probing of anatomy within and adjacent to a patient's nasal cavity.

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. In some instances, the instrument may be placed in a reprocessing tray (e.g., a metal bin or basket) and then cleaned in a surgical instrument washer. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, steam, hydrogen peroxide vapor (e.g., via a STERRAD sterilization system by Advanced Sterilization Products of Irvine, Calif.), and/or using any other suitable systems or techniques.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. An apparatus comprising:
(a) a cannula including:
(i) a proximal end,
(ii) a distal end, and
(iii) a first lumen extending from the proximal end to the distal end, wherein the cannula is formed of a rigid material; and
(b) a grip portion secured to the proximal end of the cannula, wherein the grip portion includes:
(i) a second lumen, wherein the second lumen is in communication with the first lumen,
(ii) a transversely oriented vent port, wherein the transversely oriented vent port is configured to communicate with the second lumen to thereby vent the second lumen to atmosphere,
(iii) a suction port, wherein the suction port is configured to communicate with the second lumen to thereby communicate suction to the distal end of the cannula via the first lumen, and
(iv) a guidewire port, wherein the guidewire port is configured to receive a guidewire, wherein the guidewire port is further configured to communicate with the second lumen to thereby provide a path for the guidewire to reach the first lumen.

2. The apparatus of claim 1, wherein the cannula further includes a preformed bend formed between the proximal end and the distal end.

3. The apparatus of claim 1, wherein the cannula is formed of rigid steel.

4. The apparatus of claim 1, wherein the second lumen defines a longitudinal axis, wherein the suction port is oriented obliquely relative to the longitudinal axis.

5. The apparatus of claim 4, wherein the guidewire port is coaxially aligned with the longitudinal axis.

6. The apparatus of claim 1, wherein the second lumen defines a longitudinal axis, wherein the guidewire port is oriented obliquely relative to the longitudinal axis.

7. The apparatus of claim 6, wherein the suction port is coaxially aligned with the longitudinal axis.

8. The apparatus of claim 1, wherein the second lumen defines a longitudinal axis, wherein the guidewire port is oriented obliquely relative to the longitudinal axis, wherein the suction port is oriented obliquely relative to the longitudinal axis.

9. The apparatus of claim 1, wherein the guidewire port defines a third lumen, wherein the third lumen distally terminates at the second lumen.

10. The apparatus of claim 9, wherein the vent port is located at a first longitudinal position along the second lumen, wherein the third lumen distally terminates at the second lumen at a second longitudinal position along the second lumen, wherein the second longitudinal position is distal to the first longitudinal position.

11. The apparatus of claim 1, wherein the grip portion further includes a locking feature, wherein the locking feature is configured to selectively engage a guidewire disposed in the guidewire port to thereby selectively secure the position of the guidewire relative to the grip portion by moving between a locked position and an unlocked position.

12. The apparatus of claim 11, wherein the grip portion further includes:
(i) a body, wherein the vent port is formed in the body, and
(ii) an arm extending obliquely from the body, wherein the guidewire port is located on the arm, wherein the locking feature is located on the arm.

13. The apparatus of claim 12, wherein the locking feature comprises a button, wherein the button is configured to translate transversely relative to a longitudinal axis defined by the arm to thereby transition between the locked position and the unlocked position.

14. The apparatus of claim 12, wherein the cannula, the vent port, and the arm are aligned along a shared plane.

15. The apparatus of claim 12, wherein the cannula and the vent port are aligned along a first plane, wherein the arm is aligned along a second plane, wherein the second plane is transverse to the first plane.

16. The apparatus of claim 11, wherein the locking feature is resiliently biased toward the locked position.

17. The apparatus of claim 11, wherein the locking feature is further configured to provide a fluid seal of the guidewire port when the locking feature is in the locked position.

18. The apparatus of claim 11, wherein the guidewire port defines a third lumen, wherein the locking feature defines a fourth lumen, wherein the third and fourth lumens are configured to receive the guidewire, wherein the locking feature in the unlocked position is configured to provide alignment of the third and fourth lumens, wherein the locking feature in the locked position is configured to provide non-alignment of the third and fourth lumens.

19. An apparatus comprising:
(a) a cannula including:
(i) a proximal end,
(ii) a distal end, and
(iii) a first lumen extending from the proximal end to the distal end, wherein the cannula is formed of a rigid material; and
(b) a grip portion secured to the proximal end of the cannula, wherein the grip portion includes:
(i) a second lumen, wherein the second lumen is in communication with the first lumen,
(ii) a suction port, wherein the suction port is configured to communicate with the second lumen to thereby communicate suction to the distal end of the cannula via the first lumen,
(iii) a guidewire port, wherein the guidewire port is configured to receive a guidewire, wherein the guidewire port is further configured to communicate with the second lumen to thereby provide a path for the guidewire to reach the first lumen, and
(iv) a locking feature, wherein the locking feature is configured to selectively engage a guidewire disposed in the guidewire port to thereby selectively secure the position of the guidewire relative to the grip portion.

20. An apparatus comprising:
(a) a cannula including:
(i) a proximal end,
(ii) a distal end, and
(iii) a first lumen extending from the proximal end to the distal end;
(b) a grip portion secured to the proximal end of the cannula, wherein the grip portion includes:
(i) a second lumen, wherein the second lumen is in communication with the first lumen,
(ii) a suction port, wherein the suction port is configured to communicate with the second lumen to thereby communicate suction to the distal end of the cannula via the first lumen, and
(iii) a guidewire port, wherein the guidewire port is configured to receive a guidewire, wherein the guidewire port is further configured to communicate with the second lumen to thereby provide a path for the guidewire to reach the first lumen; and
(c) a navigation guidewire configured to fit in the guidewire port, wherein the navigation guidewire is further configured to fit in the first lumen, wherein the navigation guidewire includes a sensor, wherein the sensor is configured to generate data based on a location of the sensor within a patient;
wherein the first lumen is configured to provide suction through the distal end while the navigation guidewire is disposed in the first lumen.
